(19) United States
(12) Patent Application Publication
(10) Pub. No.: US 2005/0261285 A1
(43) Pub. Date: Nov. 24, 2005

(54) Tetrahydropyran derivatives and their use as therapeutic agents

(76) Inventors: Olivier Dirat, Bishop's Stortford (GB); Jason Matthew Elliott, Felsted (GB); Janusz Jozef Kulagowski, Sawbridgeworth (GB); Simon Neil Owen, London (GB); Piotr Antoni Raubo, Bishop's Stortford (GB); Duncan Edward Shaw, Bishop's Stortford (GB); Brian John Williams, Great Dunmow (GB)

Correspondence Address: MERCK AND CO., INC, P O BOX 2000, RAHWAY, NJ, 07065-0907, US

(21) Appl. No.: 10/521,338

(22) PCT Filed: Jul. 17, 2003
(86) PCT No.: PCT/GB03/03098

§ 371 Date: Jan. 12, 2005

(30) Foreign Application Priority Data
Jul. 23, 2002 (GB) 0217068.6

(51) Int. Cl. A61K 31/541; A61K 31/513; C07D 417/14

(52) U.S. Cl. 514/227.5; 544/58.5; 544/316; 514/269

(57) ABSTRACT

The present invention relates compounds of the formula (I), wherein R¹, R², R³, R⁴, R⁵, R⁶, R⁷, R⁸, R¹⁵ and R¹⁶ represent a variety of substituents; and pharmaceutically acceptable salts thereof. The compounds are of particular use in the treatment or prevention of depression, anxiety, pain, inflammation, migaine, emesis or postherpetic neuralgia.

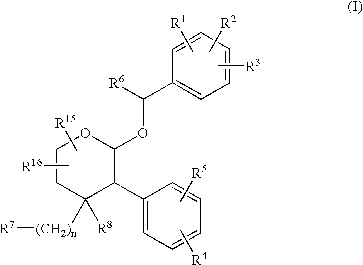

This invention relates to a class of tetrahydropyran compounds which are useful as tachykinin antagonists. More particularly, the compounds of the invention are useful as neurokinin 1 (NK-1) receptor antagonists.

International (PCT) Patent Publication Nos. WO 00/56727, published 28^{th} Sep. 2000, and WO 02/16344, published 28^{th} Feb. 2002, describe classes of tetrahydropyran derivatives and their use as NK-1 receptor antagonists. The novel compounds of the present invention are characterised by the 5- or 6-membered carbonyl or sulfonyl containing cyclic moiety represented by the R⁷ substituent.

International (PCT) Patent Publication No. WO 03/022839, published 20^{th} Mar. 2003 (after the priority date of the present invention), describes a further class of tetrahydropyran derivatives and their use as NK-1 receptor antagonists. The compounds of the present invention are novel in view of the nature of the substituents at the 4-position on the tetrahydropyran ring.

The present invention provides compounds of the formula (I): 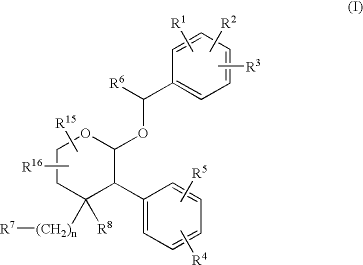
wherein
R¹ is hydrogen, halogen, C₁₋₆alkyl, C₁₋₆alkoxy, fluoroC₁₋₆alkyl, fluoroC₁₋₆alkoxy, C_{3,7}cycloalkyl, C_{3,7}cycloalkylC₁₋₄alkyl, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} each independently represent hydrogen or C₁₋₄alkyl;
R² is hydrogen, halogen, C₁₋₆alkyl, fluoroC₁₋₆alkyl or C₁₋₆alkoxy substituted by C₁₋₄alkoxy;
R³ is hydrogen, halogen or fluoroC₁₋₆alkyl;
R⁴ is hydrogen, halogen, C₁₋₆alkyl, C₁₋₆alkoxy, fluoroC₁₋₆alkyl, fluoroC₁₋₆alkoxy, hydroxy, NO₂, CN, SR^{a}, SOR^{a}, SO₂R^{a}, CO₂R^{a}, CONR^{a}R^{b}, C₂₋₆alkenyl, C₂₋₆alynyl or C₁₋₄alkyl substituted by C₁₋₄alkoxy, wherein R^{a} and R^{b} are as previously defined;
R⁵ is hydrogen, halogen, C₁₋₆alkyl, fluoroC₁₋₆alkyl or C₁₋₆alkoxy substituted by C₁₋₄alkoxy;
R⁶ represents hydrogen or a C₁₋₄alkyl group optionally substituted by a hydroxy group;
R⁷ represents a 5- or 6-membered carbonyl or sulfonyl containing cyclic group comprising from 0 to 3 nitrogen ring atoms, from 0 to 1 oxygen ring atom and from 0 to 1 sulfur ring, wherein said ring is optionally substituted at any substitutable position by one or more substituents selected from ═O, halogen, hydroxy, R¹¹, R¹², SR^{f}, SO₂R^{g}, COR^{a}, CO₂R^{a}, CONR⁹R¹⁰, -ZNR⁹R¹⁰, benzyl, C₁₋₄alkyl, hydroxyC₁₋₄alkyl, fluoroC₁₋₄alkyl, chloroC₁₋₄alkyl, C₁₋₄alkoxyC₁₋₄alkyl, C₃₋₇cycloalkyl, C₃₋₇cycloalkylC₁₋₄alkyl, C₃₋₇cycloalkoxy, C₃₋₇cycloalkoxyC₁₋₄alkyl, C₁₋₄alkoxy, fluoroC₁₋₄alkoxy, hydroxyC₁₋₄alkoxy, C₁₋₄alkoxyC₁₋₄alkoxy, aryl, arylC₁₋₄alkyl heteroaryl, heteroarylC₁₋₄alkyl or a 5- or 6-membered ring containing in the ring one oxygen atom or N(C₁₋₆alkyl), wherein R^{f} is C₁₋₄alkyl or aralkyl or aryl and R^{g} is C₁₋₄alkyl, aryl, arylC₁₋₄alkyl or NR⁹R¹⁰;
R⁸ represents hydrogen, C₁₋₆alkyl, fluoroC₁₋₆alkyl, hydroxy, C₁₋₆alkoxy, hydroxyC₁₋₆alkyl NR⁹R¹⁰, CONR⁹R¹⁰ or SO₂R^{g};
R⁹ is hydrogen, C₁₋₄alkyl, C₃₋₇cycloalkyl, C₃₋₇cycloalkylC₁₋₄alkyl, fluoroC₁₋₄alkyl C₂₋₄alkyl substituted by a C₁₋₄alkoxy or hydroxyl group, or R⁹ is a five membered or six membered nitrogen-containing heteroaromatic ring as previously defined;
R¹⁰ is hydrogen or C₁₋₄alkyl, C₃₋₇cycloalkyl, C₃₋₇cycloalkylC₁₋₄alkyl, fluoroC₁₋₄alkyl or C₂₋₄alkyl substituted by a C₁₋₄alkoxy or hydroxyl group;
or R⁹, R¹⁰ and the nitrogen atom to which they are attached form a heteroaliphatic ring of 4 to 7 ring atoms, optionally substituted by one or two groups selected from hydroxy, COR^{e}, CO₂R^{e}, C₁₋₄alkyl optionally substituted by a C₁₋₄alkoxy or hydroxyl group, or C₁₋₄alkoxy optionally substituted by a C₁₋₄alkoxy or hydroxyl group, or a five membered or six membered nitrogen-containing heteroaromatic ring as previously defined, or said heteroaliphatic ring is substituted by a spiro-fused lactone ring, and said heteroaliphatic ring optionally containing a double bond, which heteroaliphatic ring may optionally contain an oxygen or sulphur ring atom, a group S(O) or S(O)₂ or a second nitrogen atom which will be part of a NH or NR^{d} moiety, where R^{d} is C₁₋₄alkyl optionally substituted by hydroxy or C₁₋₄alkoxy;
or R⁹, R¹⁰ and the nitrogen atom to which they are attached form a non-aromatic azabicyclic ring system of 6 to 12 ring atoms;
or R⁹, R¹⁰ and the nitrogen atom to which they are attached form a heteroaliphatic ring of 4 to 7 ring atoms to which is fused a benzene ring or a five membered or six membered nitrogen-containing heteroaromatic ring optionally containing 1, 2 or 3 additional heteroatoms selected from N, O and S;
R¹¹ and R¹² each independently represent hydrogen, hydroxy, COR^{e}, CO₂R^{e}, C₁₋₄alkyl optionally substituted by a C₁₋₄alkoxy or hydroxyl group, or C₁₋₄alkoxy optionally substituted by a C₁₋₄alkoxy or hydroxyl group;
or, when they are attached to the same carbon atom, R¹¹ and R¹² may together represent ═O, ═CHCO₂R^{a}, —O(CH₂)ₘO—, —CH₂O(CH₂)ₖ—, —CH₂OCH₂C(O)—, —CH₂OCH₂CH(OH)—, —CH₂OCH₂C(CH₃)₂—, —CH₂OC(CH₃)₂CH₂—, —C(CH₃)₂OCH₂CH₂—, —CH₂C(O)OCH₂—, —OC(O)CH₂CH₂—, —C(O)OCH₂CH₂—, —C(O)OC(CH₃)₂CH₂—, —C(O)OCH₂C(CH₃)₂—, —OCH₂(CH₂)ₖ—, —OC(CH₃)₂CH₂CH₂—, —OCH₂C(CH₃)₂CH₂—, —OCH₂CH₂C(CH₃)₂—, —OCH₂CH═CHCH₂—, —OCH₂CH(OH)CH₂CH₂—, —OCH₂CH₂CH(OH)CH₂—, —OCH₂C(O)CH₂CH₂—, —OCH₂CH₂C(O)CH₂—, or a group of the formula 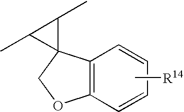
or, where they are attached to adjacent carbon atoms, R¹¹ and R¹² may together represent —OCH₂CH₂— or —OCH₂CH(OH)—, or R¹¹ and R¹² may together form a fused benzene ring;
or, R¹¹ and R¹² together form a C₁₋₂alkylene bridge across the pyrrolidine, piperidine, morpholine or piperazine ring to which they are attached;
R¹³ represents hydrogen, phenyl, benzyl, pyridyl, tetrahydropyranyl, piperidinyl, N-substituted piperidinyl (where the N-substituent is C₁₋₆alkyl), C₁₋₄alkyl, C₃₋₇cycloalkyl, C₃₋₇cycloalkylC₁₋₄alkyl, —SO₂C₁₋₄alkyl or C₂₋₄alkyl substituted by a C₁₋₄alkoxy or hydroxyl group;
R¹⁴ represents hydrogen, halogen, hydroxy, C₁₋₄alkyl, hydroxyC₁₋₄alkyl or fluoroC₁₋₄alkyl;
R¹⁵ and R¹⁶ each independently represent hydrogen, halogen, C₁₋₆alkyl, CH₂OR^{c}, oxo, CO₂R^{a} or CONR^{a}R^{b} where R^{a} and R^{b} are as previously defined and R^{c} represents hydrogen, C₁₋₆alkyl or phenyl;
Z represents a bond, C₁₋₆alkylene or C₃₋₆cycloalkylene;
k is 1, 2 or 3;
m is 1 or 2; and
n is zero, 1 or 2;
with the proviso that when n is zero and R⁸ is hydrogen, R⁷ does not represent a C-linked nitrogen-containing ring of the formula 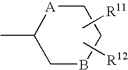
   wherein
A represents NR¹³, and B represents a bond, CH₂, NR¹³ or O, wherein one or both hydrogen atoms in said CH₂ moiety may be replaced with one or both of R¹¹ and R¹², or alternatively, one of the hydrogen atoms in said CH₂ moiety together with a hydrogen atom from an adjacent carbon are replaced by a double bond; or A is O, and B is NR¹³; and R¹¹ and R¹² together represent ═O;
   and pharmaceutically acceptable salts thereof.

One particular aspect of the present invention is the class of compounds of formula (I) and pharmaceutically acceptable salts thereof, wherein
R⁷ represents a 5- or 6-membered carbonyl or sulfonyl containing cyclic group comprising from 0 to 3 nitrogen ring atoms, from 0 to 1 oxygen ring atom and from 0 to 1 sulfur ring, wherein said ring is optionally substituted at any substitutable position by one or more substituents selected from ═O, halogen, hydroxy, R¹¹, R¹², SR^{f}, SO₂R^{g}, COR^{a}, CO₂R^{a}, CONR⁹R¹⁰, -ZNR⁹R¹⁰, benzyl C₁₋₄alkyl, hydroxyC₁₋₄alkyl, fluoroC₁₋₄alkyl chloroC₁₋₄alkyl, C₁₋₄alkoxyC₁₋₄alkyl, C₃₋₇cycloalkyl, C₃₋₇cycloalkylC₁₋₄alkyl, C₃₋₇cycloalkoxy, C₃₋₇cycloalkoxyC₁₋₄alkyl, C₁₋₄alkoxy, fluoroC₁₋₄alkoxy, hydroxyC₁₋₄alkoxy, C₁₋₄alkoxyC₁₋₄alkoxy, aryl or arylC₁₋₄alkyl wherein R^{f} is C₁₋₄alkyl or aralkyl or aryl and R^{g} is C₁₋₄alkyl, aryl arylC₁₋₄alkyl or NR⁹R¹⁰;
R¹³ represents hydrogen, benzyl, C₁₋₄alkyl, C₃₋₇cycloalkyl, C₃₋₇cycloalkylC₁₋₄alkyl, —SO₂C₁₋₄alkyl or C₂₋₄alkyl substituted by a C₁₋₄alkoxy or hydroxyl group;
   and the remaining groups are as defined above.

A preferred class of compounds of formula (I) is that wherein R¹ is hydrogen, C₁₋₄alkyl C₁₋₄alkoxy, halogen or CF₃.

Another preferred class of compounds of formula (I) is that wherein R² is hydrogen, C₁₋₄alkyl, C₁₋₄alkoxy, halogen or CF₃.

Also preferred is the class of compounds of formula (I) wherein R₃ is hydrogen, fluorine, chlorine or CF₃.

A particularly preferred class of compounds of formula (I) is that wherein R¹ is fluorine, chlorine or CF₃.

Another particularly preferred class of compounds of formula (I) is that wherein R² is hydrogen, fluorine, chlorine or CF₃.

Also particularly preferred is the class of compounds of formula (I) wherein R² is hydrogen, fluorine, chlorine or CF₃.

Preferably R¹ and R² are in the 3 and 5 positions of the phenyl ring.

More preferably R¹ is 3-fluoro or 3-CF₃.

More preferably R² is 5-fluoro or 5-CF₃.

More preferably R³ is hydrogen.

Most preferably R¹ is 3-F or 3-CF₃, R² is 5-CF₃ and R₃ is hydrogen.

A further preferred class of compound of formula (I) is that wherein R⁴ is hydrogen or fluorine, especially hydrogen.

Another preferred class of compounds of formula (I) is that wherein R⁵ is hydrogen, fluorine, chlorine or CF₃.

Preferably R⁴ is hydrogen or 3-fluoro, especially hydrogen, and R⁵ is hydrogen or 4-fluoro.

R⁶ is preferably C₁₋₄alkyl optionally substituted by hydroxy. In particular, R⁶ is preferably a methyl or hydroxymethyl group. Most especially, R⁶ is a methyl group.

A further preferred class of compounds of formula (I) is that wherein R⁷ is a cyclic group selected from the group consisting of: 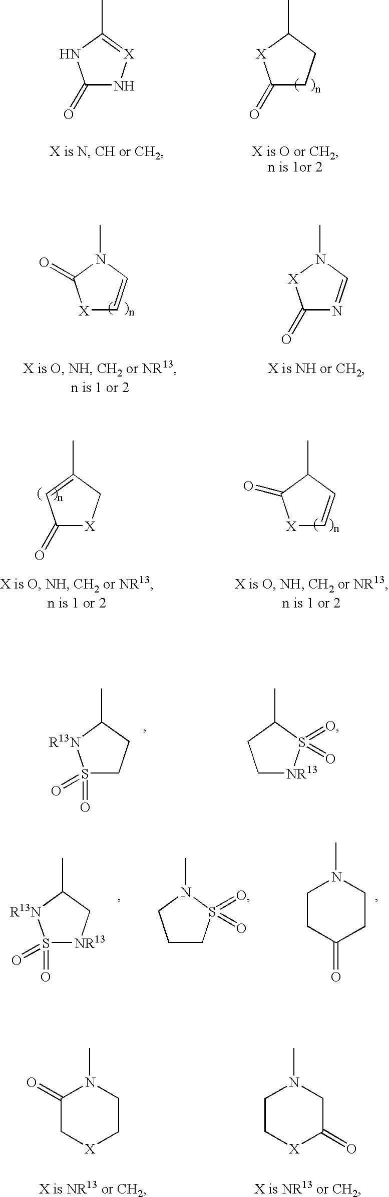 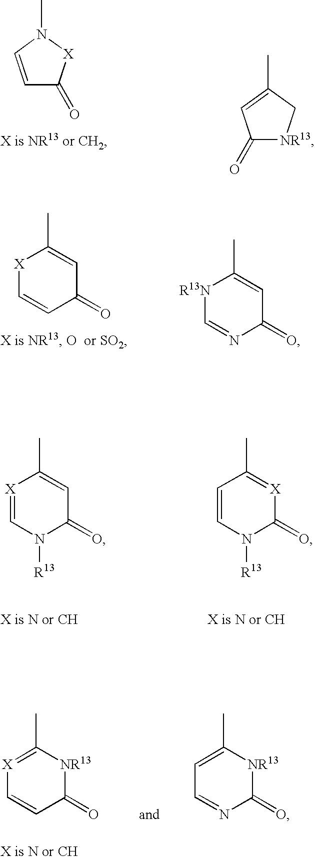
wherein R¹³ is as previously defined, and further wherein any of said cyclic groups is optionally substituted by one or more (preferably one or two) groups as previously defined.

Also preferred is the class of compound of formula (I) wherein R⁷ is a cyclic group selected from the group consisting of: 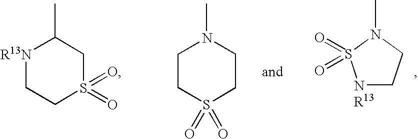
wherein R¹³ is as previously defined, and further wherein any of said cyclic groups is optionally substituted by one or more (preferably one or two) groups as previously defined.

Another preferred class of compound of formula (I) is that wherein R⁸ is hydrogen or methyl, and especially hydrogen.

Another preferred class of compounds of formula (I) is that wherein R¹² is hydrogen, hydroxy, C₁₋₂alkyl substituted by hydroxy, C₁₋₄alkoxy (especially methoxy) or CO₂R^{e} (where R^{e} is hydrogen, methyl ethyl or benzyl).

A further preferred class of compounds of formula (I) is that wherein R¹² is hydrogen or C₁₋₄alkyl (especially methyl).

Where R¹¹ and R¹² are attached to the same carbon atom they may, in particular, together represent —C(O)OCH₂CH₂—.

In a further preferred class of compounds of formula (I), R¹³ preferably represents hydrogen, methyl or ethyl.

Another preferred class of compound of formula (I) is that wherein one of R¹⁵ and R¹⁶ is hydrogen, and especially wherein R¹⁵ and R¹⁶ are both hydrogen atoms.

A further preferred class of compound of formula (I) is that wherein n is zero or 1, and especially wherein n is zero.

In the definition of the group —NR⁹R¹⁰, R⁹ may aptly be a C₁₋₄alkyl group or a C₂₋₄alkyl group substituted by a hydroxyl or C₁₋₂alkoxy group, R¹⁰ may aptly be a C₁₋₄alkyl group or a C₂₋₄alkyl group substituted by a hydroxyl or C₁₋₂alkoxy group, or R⁹ and R¹⁰ may be linked so that, together with the nitrogen atom to which they are attached, they form an azetidinyl, pyrrolidinyl, piperidinyl, morpholino, thiomorpholino, piperazino or piperazino group substituted on the nitrogen atom by a C₁₋₄alkyl group or a C₂₋₄alkyl group substituted by a hydroxy or C₁₋₂alkoxy group. Particularly preferred heteroaliphatic rings formed by —NR⁹R¹⁰ are azetidine, pyrolidine, piperidine, morpholine, piperazine and N-methylpiperazine, and especially piperidine.

Where the group NR⁹R¹⁰ represents a heteroaliphatic ring of 4 to 7 ring atoms substituted by two groups, the first substituent, where present, is preferably selected from hydroxy, CO₂R^{e} (where R^{e} is hydrogen, methyl ethyl or benzyl), or C₁₋₂alkyl substituted by hydroxy. Where present, the second substituent is preferably a methyl group. Where two substituents are present, said substituents are preferably attached to the same carbon atom of the heteroaliphatic ring.

Where the group NR⁹R¹⁰ represents a heteroaliphatic ring of 4 to 7 ring atoms substituted by a spiro-fused lactone ring, particularly preferred examples are:

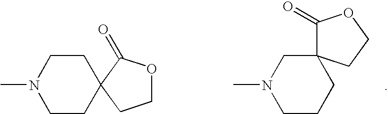

Where the group NR⁹R¹⁰ represents a heteroaliphatic ring of 4 to 7 ring atoms and said ring contains a double bond, a particularly preferred group is 3-pyrroline.

Where the group NR⁹R¹⁰ represents a non-aromatic azabicyclic ring system, such a system may contain between 6 and 12, and preferably between 7 and 10, ring atoms. Suitable rings include 5-azabicyclo[2.1.1]hexyl, 5-azabicyclo[2.2.1]heptyl, 6-azabicyclo[3.2.1]octyl, 2-azabicyclo[2.2.2]octyl, 6-azabicyclo[3.2.2]nonyl, 6-azabicyclo[3.3.1]nonyl, 6-azabicyclo[3.3.2]decyl, 7-azabicyclo[4.3.]decyl, 7-azabicyclo[4.4.1]undecyl and 8-azabicyclo[5.4.1]dodecyl, especially 5-azabicyclo[2.2.1]heptyl and 6-azabicyclo[3.2.1]octyl.

Where the group NR⁹R¹⁰ represents a heteroaliphatic ring of 4 to 7 ring atoms to which is fused a benzene ring or a five membered or six membered nitrogen-containing heteroaromatic ring ring optionally containing 1, 2 or 3 additional heteroatoms selected from N, O and S, said heteroaromatic ring is preferably a five-membered ring, in particular a pyrrole, imidazole or triazole ring, a nitrogen atom of which is preferably included in the heteroaliphatic ring. Suitable examples of such fused ring systems include

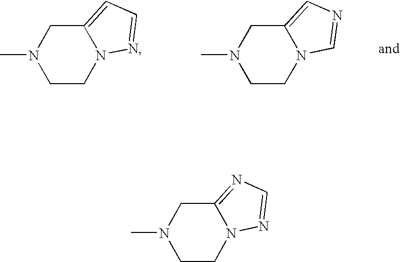

Particularly suitable moieties NR⁹R¹⁰ include those wherein NR⁹R¹⁰ is amino, methylamino, dimethylamino, diethylamino, azetidino, pyrrolidino, piperidino, morpholino and piperazino.

Favourably Z is a bond or contains 1 to 4 carbon atoms and most favourably 1 to 2 carbon atoms. A particularly favourable group Z is —CH₂—. The group -ZNR⁹R¹⁰, as a substituent on a heteroaromatic ring, is preferably CH₂N(CH₃)₂.

One favoured group of compounds of the present invention are of the formula (Ia) and pharmaceutically acceptable salts thereof: 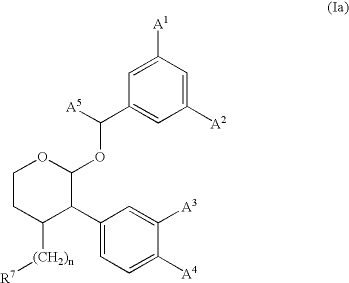
wherein
A¹ is fluorine or CF₃;
A² is fluorine or CF₃;
A³ is fluorine or hydrogen;
A⁴ is fluorine or hydrogen;
A⁵ is methyl; and
R⁷ and n are as defined in relation to formula (I).

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl n-propyl, i-propyl n-butyl, s-butyl and t-butyl. Examples of suitable alkoxy groups include methoxy, ethoxy, n-propoxy, i-propoxy, n-butoxy, s-butoxy and t-butoxy.

As used herein, the terms “fluoroC₁₋₆alkyl” and fluoroC₁₋₆alkoxy” means a C₁₋₆alkyl or C₁₋₆alkoxy group in which one or more (in particular, 1 to 3) hydrogen atoms have been replaced by fluorine atoms. Similarly, the term “fluoroC₁₋₄alkyl” means a C₁₋₄alkyl group in which one or more (in particular 1 to 3) hydrogen atoms have been replaced by fluorine atoms. Particularly preferred are fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CFs, OCFA and OCH₂CF₃.

The cycloalkyl groups referred to herein may represent, for example, cyclopropyl, cyclobutyl, cyclopentyl or cyclohexyl. A suitable cycloalkylalkyl group may be, for example, cyclopropylmethyl.

Similarly cycloalkoxy groups referred to herein may represent, for example, cyclopropoxy or cyclobutoxy.

As used herein, the terms “alkenyl” and “alkynyl” as a group or part of a group means that the group is straight or branched. Examples of suitable alkenyl groups include vinyl and allyl. A suitable alkynyl group is propargyl.

As used herein, the term “aryl” as a group or part of a group means an aromatic radical such as phenyl, biphenyl or naphthyl, wherein said phenyl, biphenyl or naphthyl group may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)ₘO—. Preferably said phenyl, biphenyl or naphthyl group is optionally substituted by one or two substituents, especially none or one. Particularly preferred substituents include fluorine, chlorine, bromine, C₁₋₄alkyl (especially methyl), C₁₋₄alkoxy (especially methoxy), trifluoromethyl, trifluormethoxy or vinyl.

Reference herein to “an optionally substituted five or six-membered nitrogen-containing heteroaromatic ring optionally containing 1, 2 or 3 additional heteroatoms selected from N, O and S”, is preferably reference to a heteroaromatic ring is selected from pyrrole, pyridine, pyrazole, imidazole, oxazole, isoxazole, thiazole, isothiazole, pyrazine, pyrimidine, pyridazine, triazole, oxadiazole, thiadiazole, triazine, and tetrazole.

Suitable 5- or 6-membered cyclic ethers include optionally substituted tetrahydropyran and tetrahydrofuran rings.

When used herein the term “halogen” means fluorine, chlorine, bromine and iodine. The most apt halogens are fluorine and chlorine of which fluorine is preferred, unless otherwise stated.

In a further aspect of the present invention, the compounds of formula (I) may be prepared in the form of a pharmaceutically acceptable salt, especially an acid addition salt.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. Salts of amine groups may also comprise quaternary ammonium salts in which the amino nitrogen atom carries a suitable organic group such as an alkyl alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula (I) and salts thereof, for example, hydrates.

The compounds according to the invention have at least three asymmetric centres, and may accordingly exist both as enantiomers and as diastereoisomers. It is to be understood that all such isomers and mixtures thereof are encompassed within the scope of the present invention.

The preferred compounds of the formula (I) and (Ia) will have the stereochemistry of the 3-, 4- and 5-positions as shown in formulae (Ib) and (Ic)

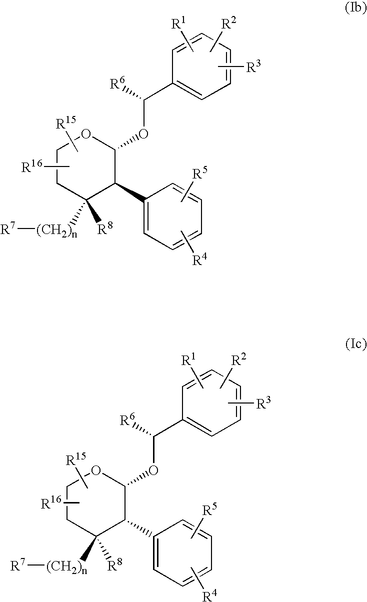

It will be appreciated that the preferred definitions of the various substituents recited herein may be taken alone or in combination and, unless otherwise stated, apply to the generic formula for compounds of the present invention as well as to the preferred classes of compound represented by formula (Ia), formula (Ib) and formula (Ic).

The present invention further provides pharmaceutical compositions comprising one or more compounds of formula (I) in association with a pharmaceutically acceptable carrier or excipient.

Preferably the compositions according to the invention are in unit dosage forms such as tablets, pills, capsules, powders, granules, solutions or suspensions, or suppositories, for oral, parenteral or rectal administration, or administration by inhalation or insufflation. Oral compositions such as tablets, pills, capsules or wafers are particularly preferred.

A more detailed description of pharmaceutical compositions that are suitable for the formulation of compounds of the present invention is disclosed in U.S. Pat. No. 6,071,927, the content of which is incorporated herein by reference (see in particular, column 8, line 50 to column 10, line 4).

The present invention further provides a process for the preparation of a pharmaceutical composition comprising a compound of formula (I), which process comprises bringing a compound of formula (I) into association with a pharmaceutically acceptable carrier or excipient.

The compounds of formula (I) are of value in the treatment of a wide variety of clinical conditions which are characterised by the presence of an excess of tachykinin, in particular substance P, activity. A comprehensive listing of clinical conditions, uses and methods of treatment for which the compounds of the present invention will be useful is disclosed in U.S. Pat. No. 6,071,927, the content of which is incorporated herein by reference (see, in particular, column 10, line 14 to column 22, line 18).

In particular, the compounds of the present invention are useful in the treatment of a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; and anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

The compounds of the present invention are also particularly useful in the treatment of nociception and pain. Diseases and conditions in which pain predominates, include soft tissue and peripheral damage, such as acute trauma, osteoarthritis, rheumatoid arthritis, musculo-skeletal pain, particularly after trauma, spinal pain, myofascial pain syndromes, headache, migraine, episiotomy pain, and burns.

The compounds of the present invention are also particularly useful in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; in the treatment of inflammatory diseases such as inflammatory bowel disease, psoriasis, fibrositis, osteoarthritis, rheumatoid arthritis, pruritis and sunburn; and in the treatment of allergic disorders such as eczema and rhinitis.

The compounds of the present invention are also particularly useful in the treatment of gastrointestinal (GI) disorders, including inflammatory disorders and diseases of the GI tract such as ulcerative colitis, Crohn's disease and irritable bowel syndrome.

The compounds of the present invention are also particularly useful in the treatment of emesis, including acute, delayed or anticipatory emesis, such as emesis induced by chemotherapy, radiation, toxins, pregnancy, vestibular disorders, motion, surgery, migraine, and variations in intercranial pressure. Most especially, the compounds of formula (I) are of use in the treatment of emesis induced by antineoplastic (cytotoxic) agents, including those routinely used in cancer chemotherapy; by radiation including radiation therapy such as in the treatment of cancer; and in the treatment of post-operative nausea and vomiting.

The excellent pharmacological profile of the compounds of the present invention offers the opportunity for their use in therapy at low doses thereby minimising the risk of unwanted side effects.

In the treatment of the conditions associated with an excess of tachykinins, a suitable dosage level is about 0.001 to 50 mg/kg per day, in particular about 0.01 to about 25 mg/kg, such as from about 0.05 to about 10 mg/kg per day.

For example, in the treatment of conditions involving the neurotransmission of pain sensations, a suitable dosage level is about 0.001 to 25 mg/kg per day, preferably about 0.005 to 10 mg/kg per day, and especially about 0.005 to 5 mg/kg per day. The compounds may be administered on a regimen of 1 to 4 times per day, preferably once or twice per day.

In the treatment of emesis, a suitable dosage level is about 0.001 to 10 mg/kg per day, preferably about 0.005 to 5 mg/kg per day, and especially 0.01 to 3 mg/kg per day. The compounds may be administered on a regimen of 1 to 4 times per day, preferably once or twice per day.

In the treatment of psychiatric disorders, a suitable dosage level is about 0.001 to 10 mg/kg per day, preferably about 0.005 to 5 mg/kg per day, and especially 0.01 to 3 mg/kg per day. The compounds may be administered on a regimen of 1 to 4 times per day, preferably once or twice per day.

It will be appreciated that the amount of a compound of formula (1) required for use in any treatment will vary not only with the particular compounds or composition selected but also with the route of administration, the nature of the condition being treated, and the age and condition of the patient, and will ultimately be at the discretion of the attendant physician.

As used herein, the term “treatment” includes prophylactic use to prevent the occurrence or recurrence of any of the aforementioned conditions.

According to a general process (A), compounds of formula (I), in which R⁷ is an N-linked cyclic group, may be prepared by the reaction of a compound of formula (II) 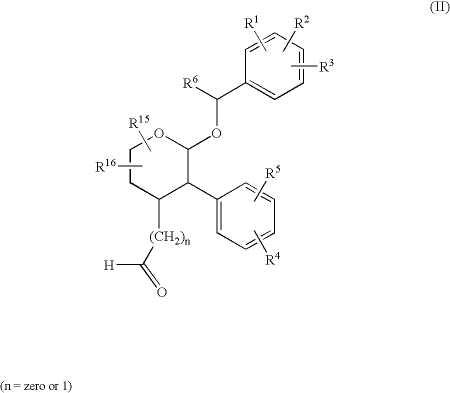
with an amine of the formula HNR⁹R¹⁰ in the presence of a reducing agent, for example, sodium triacetoxyborohydride or sodium cyanoborohydride. The reaction is conveniently effected in a suitable solvent such as a halogenated hydrocarbon, for example, 1,2-dichloroethane, conveniently at about room temperature.

Compounds of formula II) may be prepared by oxidation of a compound of formula (III)

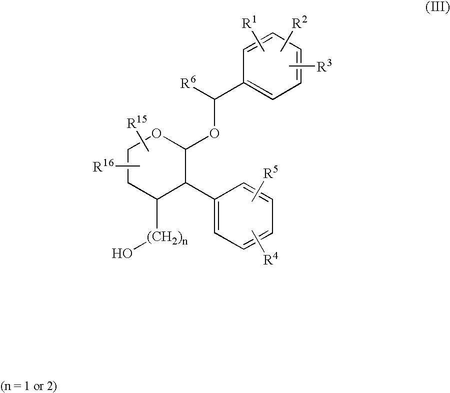

The reaction is conveniently effected under conventional conditions suitable for the oxidation of a primary alcohol to an aldehyde without further oxidation to the carboxylic acid, for example, using Dess-Martin periodinane in a suitable solvent such as a halogenated hydrocarbon, for example, dichloromethane, conveniently at about room temperature.

Compounds of formula (III) may be prepared by reaction of a compound of formula (V) 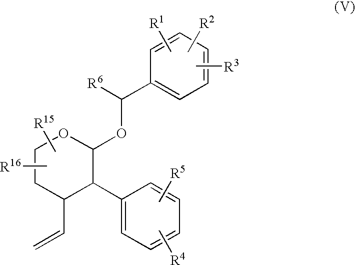
with ozone, followed by a reaction with a reducing agent such as sodium borohydride (n is 1), or by reaction with a reducing agent such as borane.tetrahydrofuran complex, followed by hydrogen peroxide in the presence of a base such as sodium hydroxide.

According to another general process (B), compounds of formula (I) may be prepared by the reaction of a compound of formula (VI) 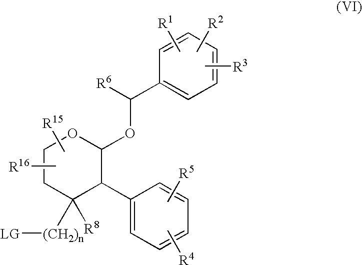
wherein LG is a suitable leaving group such as an alkyl- or arylsulfonyloxy group (e.g. mesylate or tosylate) or a halogen atom (e.g. bromine, chlorine or iodine); by reaction with an appropriate reactant to introduce a cyclic group as defined in relation to formula (I).

A particularly preferred compound of formula (VI) is that wherein the group LG is mesylate—i.e. the group —OSO₂CH₃.

According to another general process (C), compounds of formula (I) may be prepared by the reaction of a compound of formula (VII) with a compound of formula (VIII) 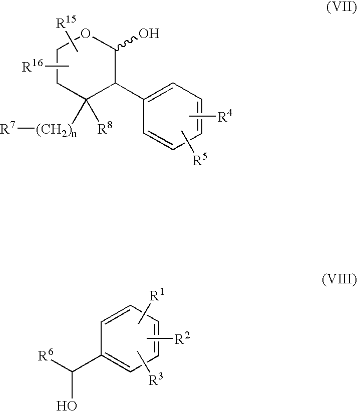
preferably in the presence of a resin catalyst such as Amberlyst™ 15, and 3 Angstrom molecular sieves.

The reaction is conveniently effected in a suitable solvent such as a halogenated hydrocarbon, for example, dichloromethane, conveniently at room temperature.

According to another general process (C), compounds of formula (I) wherein R⁸ is other than hydrogen, may be prepared by the reaction of a compound of formula (XIV) 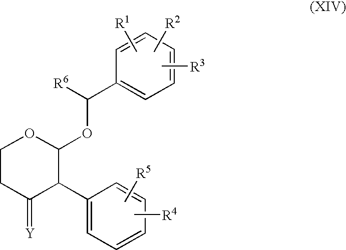
wherein Y is a suitable heteroatom or group such as an alkyl- or arylsulfinylimino group or an alkyl- or arylsulfonylimino group or an oxygen atom; by reaction with an appropriate nuclephilic reactant to introduce a R⁷—(CH₂)ₙ group as defined in relation to formula (I).

Compounds of formula (XIV) may be prepared by methods well known to one of ordinary skill in the art or by methods analogous to those described herein.

Compounds of formula (VII) may be prepared by the reduction of a compound of formula (IX) 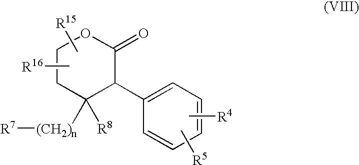
using conventional conditions such as sodium borohydride in the presence of a transition metal catalyst such as cerium chloride hexahydrate, in a solvent such as alcohol, for example, ethanol; or using DIBAL in a solvent such as a halogenated hydrocarbon, for example, dichloromethane.

Compounds of formula (VIII) may be prepared from a compound of formula (X) 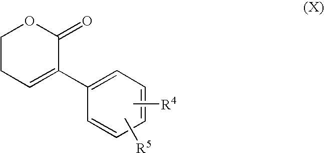
by reaction with a vinyl Grignard reagent such as R⁷(CH₂)ₙMgBr, preferably in the presence of copper(I)iodide, and a suitable solvent such as an ether, for example, tetrahydrofuran. This reaction is effected at reduced temperature, for example, below −40° C. and preferably at −78° C.

Compounds of formula (VI) and (X) are either known compounds or may be prepared by methods analogous to those described herein.

Compounds of formula (VI) may be prepared by conventional methods from, for example, a corresponding compound of formula (I) in which R⁷ is a hydroxyl group. Thus, for example, when LG is a mesylate group a corresponding compound of formula (I) in which R⁷ is hydroxyl may be reacted with methanesulfonyl chloride in the presence of a base, such as triethylamine. The reaction is conveniently effected in a solvent such as a halogenated hydrocarbon, for example, dichloromethane.

It will be appreciated that the general methodology described above may be adapted, using methods that are readily apparent to one of ordinary skill in the art, in order to prepare further compounds of the present invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The exemplified compounds of this invention were tested by the methods set out at pages 36 to 39 of International Patent Specification No. WO 93/01165. The compounds were found to be active with IC₅₀ at the human NK₁ receptor of less than 100 nM on said test method.

The following non-limiting Examples serve to illustrate the preparation of compounds of the present invention:

### Description 1

### 4-Benzyloxycarbonyl-1-[((2R,3R,4R)-2-{(1R)-1-[3,5-bis(trifluoromethyl)phenyl]ethoxy}tetrahydro-3-(4-fluorophenyl)-2H-pyran-4-yl)methyl]-piperazinone

(2R,3R,4R)-2-[(1R)-1-[3,5-Bis(trifluoromethyl)phenyl]ethoxy]-3-(4-fluorophenyl)tetrahydro-2H-pyran-4-methyl methanesulfonate (WO 00/56727-A1; 2.32 g, 4.26 mmol) was added to a solution of 4-benzyloxycarbonylpiperazin-2-one (1.30 g, 5.54 mmol) and sodium hydride (60% dispersion in mineral oil, 170 mg, 4.26 mmol) in N,N-dimethylformamide (25 mL) and the mixture was stirred at 50° C. for 16 hours. Further sodium hydride (60% dispersion in mineral oil, 85.2 mg, 2.13 mmol) was added the mixture was stirred at 50° C. for 1.5 hours. The mixture was cooled and the solvent was evaporated under reduced pressure. Water (50 mL) was added and the mixture was extracted with ethyl acetate (3×100 mL). The combined organic fractions were washed with brine (150 mL), dried (MgSO₄) and the solvent was evaporated under reduced pressure. The residue was purified by flash column chromatography on silica gel, eluting with hexane/EtOAc (70:30 increasing to 0:100) to give the title compound (2.06 g, 71%). m/z (ES⁺) 683 (M+1), 425 (M+1-C₁₀H₈F₆O).

### Description 2

### (2R,3S,4S)-2-{(1R)-1-[3,5-Bis(trifluoromethyl)phenyl]ethoxy}tetrahydro-3-(4-fluorophenyl)-2H-pyran-4-carboxaldehyde

A stirred cooled (−80° C.) solution of (2R,3S,4R)-2-[(1R)-1-[3,5-bis(triiluoromethyl)phenyl]ethoxy]-4-ethenyl-3-(4-fluorophenyl)tetrahydro-2H-pyran (WO 03/22839-A1; 2.35 g, 5.08 mmol) in methanol (15 mL) and dichloromethane (15 mL) was purged with oxygen. Ozone was bubbled through the solution until a blue coloration formed. The solution was purged with oxygen and then with nitrogen. Dimethylsulfide (8 mL, 0.109 mol) was added and the solution was stirred at room temperature for 16 hours. The solvent was evaporated under reduced pressure and the residue was purified by column chromatography on silica gel, eluting with hexane/EtOAc (100:0 increasing to 90:10), to give the title compound. ¹H NM (400 MHz, CDCl₃) δ 1.46 (3H, d J 6.6 Hz), 1.83 (1H, dddd J 13.2, 12.5, 12.5, 5 Hz), 1.95 (1H, dm, J 13.4 Hz), 3.09 (1H, dd, J 12.4, 3.2 Hz), 3.46 (1H, apparent tdd, J 12.1, 3.9, 2.5 Hz), 3.8 (1H, ddd, J 11.3, 5, 1.4 Hz), 4.03 (1H, ddd, J 12.7, 11.6, 2.8 Hz), 4.50 (1H, d, J 3.2 Hz), 4.89 (1H, q, J 6.6 Hz), 7.00 (2H, t, J 8.6 Hz), 7.23-7.20 (4H, m), 7.64 (1H, s), and 9.45 (1H, d, J 2.4 Hz).

### Description 3

### (2R,3R,4R)-2-{(1R)-1-[3,5-Bis(trifluoromethyl)phenyl]ethoxy}tetrahydro-4[(2R or S)-oxiranyl]-3-phenyl-2H-pyran; and (2R,3R,4R)-2-{(1R)-1-[3,5-Bis(trifluoromethyl)phenyl]ethoxy}tetrahydro-4-[(2S or R)-oxiranyl]-3-phenyl-2H-pyran (Isomers A and B)

Dimethylsulfoxide (10 mL) was added to sodium hydride (60% dispersion in mineral oil, 385 mg, 9.6 mmol) and the mixture was stirred at room temperature for 30 minutes. Tetrahydrofuran (20 mL) was added and the mixture was cooled to −10° C. Trimethylsulfonium iodide (2.13 g, 10.4 mmol) in dimethylsulfoxide (10 mL) was added and the mixture was stirred at 0° C. for 10 minutes. (2R,3R,4R)-2-{(1R)-1-[3,5-Bis(trifluoromethyl)phenyl]ethoxy}tetrahydro-3-phenyl-2H-pyran-4-carboxaldehyde (WO 00/56727-A1; 3.58 g, 8.0 mmol) in tetrahydrofuran (10 mL) was added and the mixture was stirred at 0° C. for 30 minutes, then at room temperature for 30 minutes. Water (100 mL) was added and the mixture was extracted with ethyl acetate (3×100 mL). The combined organic fractions were washed with water (4×100 mL) and brine (100 mL), dried (MgSO₄) and the solvent was evaporated under reduced pressure. The residue was purified by column chromatography on silica gel, eluting with hexane/EtOAc (85:15 increasing to 80:20), to give:
(2R, 3R, 4R)-2-{(1R)-1-[3,5-bis(trifluoromethyl)phenyl]ethoxy}tetrahydro-4-[(2R or S)-oxiranyl]-3-phenyl-2H-pyran (Isomer A; single diastereoisomer; epoxide stereochemistry unassigned) as a colorless oil (1.37 g, 37%); ¹H NMR (500 MHz, CDCl₃) δ 7.67 (1H, s), 7.23 (5H, m), 7.01 (2H, m), 4.97 (1H, q, J 6.6 Hz), 4.28 (1H, d, J 8.4 Hz), 4.16 (1H, br d, J 11 Hz), 3.53 (1H, br t, J 11 Hz), 2.65 (1H, dd, J 11.6, 8.4 Hz), 2.60 (1H, m), 2.34 (1H, t, J 4.5 Hz), 1.95 (1H, dd, J 4.5, 2.7 Hz), 1.84 (1H, br d, J 11 Hz), 1.68 (1H, m), 1.57 (1H, m), and 1.37 (3H, d, J 6.6 Hz); and
(2R, 3R, 4R)-2-{(1R)-1-[3,5-bis(trifluoromethyl)phenyl]ethoxy}tetrahydro-4-[(2S or R)-oxiranyl]-3-phenyl-2H-pyran (Isomer B; single diastereoisomer; epoxide stereochemistry unassigned) as a colorless oil (0.51 g, 14%); ¹H NMR (500 MHz, CDCl₃) δ 7.67 (1H, s), 7.27-7.20 (5H, m), 7.08 (2H, m), 4.96 (1H, q, J 6.6 Hz), 4.25 (1H, d, J 8.3 Hz), 4.13 (1H, br d, J 12 Hz), 3.54 (1H, br t, J 12 Hz), 2.68 (1H, m), 2.61 (1H, dd, J 11.5, 8.3 Hz), 2.50 (1H, t, J 4.6 Hz), 2.46 (1H, dd, J 4.6, 2.8 Hz), 2.03 (1H, m), 1.60 (1H, br d, J 12 Hz), 1.49 (1H, m), and 1.37 (3H, d, J 6.6 Hz);
   and a 1:1 mixture of Isomer A and Isomer B (1.16 g, 31%).

### Description 4

### (2R,3R,4R,αR or S)-α-{[(2-Hydroxyethyl)thio]methyl}-2-{(1R)-1-[3,5-bis(trifluoromethyl)phenyl]ethoxy}tetrahydro-3-phenyl-2H-pyran-4-methanol; and (2R,3R,4R,αS or R)-α-{[(2-Hydroxyethyl)thio]methyl}-2{(1R)-1-[3,5-bis(trifluoromethyl)phenyl]ethoxy tetrahydro-3-phenyl-2H-pyran-4-methanol

2-Mercaptoethanol (0.70 mL, 0.78 g, 10 mmol) was added to a degassed mixture of (2R,3R,4R)-2-{(1R) 1-[3,5-bis(trifluoromethyl)phenyl]ethoxy}tetrahydro-4-[(2R or S)-oxiranyl]-3-phenyl-2H-pyran and (2R,3R,4R)-2-{(1R)-1-[[3,5bis(trifluoromethyl)phenyl]ethoxy}tetrahydro-4-[(2S or R)-oxiranyl]-3-phenyl-2H-pyran (Description 3; 1:1 mixture of diastereoisomers, 0.46 g, 1 mmol) and potassium hydroxide (0.56 g, 10 mmol) in propan-2-ol (10 mL) and the mixture was heated under reflux for 4 hours. The mixture was cooled and the solvent was evaporated under reduced pressure. Aqueous sodium hydroxide (1M, 20 mL) was added and the mixture was extracted with diethyl ether (3×20 mL). The combined organic fractions were washed with aqueous sodium hydroxide (1M, 2×20 mL) and brine (20 mL), dried (MgSO₄) and the solvent was evaporated under reduced pressure to give the title compound (1:1 mixture of alcohol epimers) as a yellow oil (0.54 g, 100%). ¹H NMR (500 MHz, CDCl₃) δ 7.66 (1H, s), 7.26-7.23 (3H, m), 7.18 and 7.15 (2H, each s), 7.05 (2H, m), 4.95 (1H, m), 4.25 and 4.23 (1H, each d, J 8.3 Hz), 4.18 (1H, m), 3.62-3.53 (3H, m), 3.30 (1H, m), 2.90-1.51 (10H, m), and 1.36 (3H, d, J 6.6 Hz).

### Description 5

### (2R,3R,4R,αR or S)-α-{[(2-Hydroxyethyl)sulfonyl]methyl}-2-{(1R)-1-[3,5-bis(trifluoromethyl)phenyl]ethoxy}tetrahydro-3-phenyl-2H-pyran-4-methanol; and (2R,3R,4R,αS or R)-α-{[(2-Hydroxyethyl)sulfonyl]methyl}-2-{(1R)-1-[3,5-bis(trifluoromethyl)phenyl]ethoxy}tetrahydro-3-phenyl-2H-pyran-4-methanol

3-Chlorobenzenecarboperoxoic acid (77%, 268 mg, 1.2 mmol) was added to a solution of (2R,3R,4R,αR or S)-α-{[(2-hydroxyethyl)thio]methyl}-2-{(1R)-1-3,5-bis(trifluoromethyl)phenyl]ethoxy}tetrahydro-3-phenyl-2H-pyran-4-methanol and (2R,3R,4R,αS or R)-α-{[(2-hydroxyethyl)thio]methyl}-2-{(1R)-1-[3,5-bis(trifluoromethyl)phenyl]ethoxy}tetrahydro-3-phenyl-2H-pyran-4-methanol (Description 4; 1:1 mixture of alcohol epimers, 268 mg, 0.5 mmol) and sodium hydrogen carbonate (125 mg, 1.5 mmol) in dichloromethane (10 mL) and the mixture was stirred at room temperature for 1 hour. Saturated aqueous sodium hydrogen carbonate (10 mL) was added and the mixture was extracted with dichloromethane (3×20 mL). The combined organic fractions were washed with saturated aqueous potassium carbonate (20 mL), dried (MgSO₄) and the solvent was evaporated under reduced pressure to give the title compound (1:1 mixture of alcohol epimers) as a colorless foam (276 mg, 97%). ¹H NMR (500 MHz, CDCl₃) δ 7.66 (1H, s), 7.26 (3H, m), 7.18 and 7.15 (2H, each s), 7.09-7.04 (2H, m), 4.94 (1H, m), 4.25 an 4.24 (1H, each d, J 8.2 Hz), 4.19 (1H, m), 4.01-3.91 (3H, m), 3.54 (1H, m), 3.32-2.92 (4H, m), 2.88 and 2.54 (1H, each dd, J 11.5, 8.2 Hz), 2.22-1.46 (5H, m), and 1.36 (3H, d, J 6.6 Hz).

### Description 6

### (2R,3R,4R)-2-[(1R)-1-[3,5-Bis(trifluoromethyl)phenyl]ethoxyl]tetrahydro-3-phenyl-2H-pyran-4-methyl Benzenesulfonate

Benzenesulfonyl chloride (1.036 L, 1.434 kg, 8.12 mol) was added slowly to a stirred, cooled (−13° C.) solution of (2R,3R,4R)-2-[(1R)-1-[3,5-bis(trifluoromethyl)phenyl]ethoxy]tetrahydro-3-phenyl-2H-pyran-4-methanol (WO 00/56727-A1; 2.60 kg, 5.80 mol) and 1,4-diazabicyclo[2.2.2]octane (1.041 kg, 9.28 mol) in ethyl acetate (26 L) and the resulting slurry was allowed to warm to 0° C. Water (26 L) was added and the mixture was stirred at 20° C. to 25° C. for 90 minutes. The layers were separated and the organic layer was washed with hydrochloric acid (1M, 26 L) and aqueous sodium hydrogen carbonate (0.5M, 26 L). The solvent was evaporated under reduced pressure to give the title compound.

¹H NMR (400 MHz, CDCl₃) δ 7.75 (2H, m), 7.66 (1H, br s), 7.62 (1H, m), 7.48 (2H, m), 7.27-7.14 (5H, m), 6.89 (2H, m), 4.93 (1H, q, J 6.6 Hz), 4.20 (1H, d, J 8.3 Hz), 4.12 (1H, ddd, J 11.9, 4.6, 1.8 Hz), 3.81 (1H, dd, J 9.8, 3.1 Hz), 3.62 (1H, dd, J 9.8, 6.9 Hz), 3.52 (1H, td, J 11.9, 2.5 Hz), 2.51 (1H, dd, J 11.7, 8.3 Hz), 2.10 (1H, m), 1.77 (1H, m), 1.64 (1H, m), and 1.34 (3H, d, J 6.6 Hz). ¹³C NMR (100 MHz, CDCl₃) δ 145.8, 137.7, 136.0, 133.9, 131.7 (q, J_{CF} 33.2 Hz), 129.4, 128.9, 128.0, 127.6, 126.4, 123.3 (q, J_{CF} 272.8 Hz), 121.6 (m), 102.4, 73.9, 72.0, 64.7, 49.9, 39.9, 28.3, and 24.6.

### Description 7

### (2R,3R,4R)-4-(Azidomethyl)-2-[(1R)-1-[3,5-bis(trifluoromethyl)phenyl]-ethoxy]tetrahydro-3-phenyl-2H-pyran

Sodium azide (0.165 g, 2.55 mmol) was added to a solution of (2R,3R,4R)-2-[(1R)-1-[3,5-bis(trifluoromethyl)phenyl]ethoxy]tetrahydro-3-phenyl-2H-pyran-4-methyl benzenesulfonate (Description 6; 0.5 g, 0.85 mmol) in N,N-dimethylformamide (10 mL) and the mixture was stirred at 50° C. for 3 hours. The mixture was cooled and diethylether (50 mL) was added. The mixture was washed with water (×5), the organic layer was dried (MgSO₄) and the solvent was evaporated under reduced pressure to give the title compound (0.4 g, 100%). ¹H NMR (400 MHz, CDCl₃) δ 1.36 (3H, d, J 6.7 Hz), 1.59-1.69 (1H, m), 1.77-1.79 (1H, m), 1.93-2.04 (3H, m), 2.56 (1H, dd, J 8.4, 11.5 Hz), 2.94 (1H, dd, J 7.4, 12.1 Hz), 3.14 (1H, dd, J 3.1, 12.1 Hz), 3.51-3.58 (1H, m), 4.09-4.19 (2H, m), 4.23 (1H, d, J 8.2 Hz), 4.97 (1H, q, J 6.5 Hz), 7.01-7.04 (2H, m), 7.17 (2H, s), 7.23-7.26 (3H, m), and 7.66 (1H, s).

### Description 8

### (2R,3R,4R)-2-[(1R)-1-[3,5-bis(trifluoromethyl)pheny]ethoxy]tetrahydro-3-phenyl-2H-pyran-4-methylamine

Palladium on carbon (10%, 50 mg) was added to a solution of (2R,3R,4R)-4-(azidomethyl)-2-[(1R)-1-[3,5-bis(trifluoromethyl)phenyl]ethoxy]tetrahydro-3-phenyl-2H-pyran (Description 7; 0.4 g, 0.85 mmol) in tetrahydrofuran (10 mL) and the mixture was stirred under hydrogen (1 atm.) for 1 hour. The mixture was filtered through Celite™ and the solvent was evaporated under reduced pressure to give the title compound (0.38 g, 100%). ¹H NMR (400 MHz, CDCl₃) δ 1.36 (3H, d, J 6.7 Hz), 1.48-1.65 (1H, m), 1.78-1.82 (2H, m), 2.30-2.36 (1H, m), 2.44-2.52 (2H, m), 3.52-3.60 (1H, m), 4.09-4.27 (2H, m), 4.96 (1H, q, J 6.5 Hz), 7.01-7.04 (2H, m), 7.17 (2H, s), 7.22-7.26 (3H, m), and 7.66 (1H, s).

### EXAMPLE 1

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]piperazinone Hydrochloride

Palladium on carbon (10%, 240 mg) was added to a solution of 4-benzyloxycarbonyl-1-[(2R,3R,4R)-2-{(1R)-1-[3,5-bis(trifluoromethyl)phenyl]-ethoxy}tetrahydro-3-(4-fluorophenyl)-2H-pyran-4-yl)methyl]piperazinone (Description 1; 2.03 g, 2.98 mmol) in ethanol (20 mL) and the mixture was shaken under hydrogen (50 psi) for 2 hours. Further palladium on carbon (10%, 270 mg) was added and the mixture was shaken under hydrogen (50 psi) for a further 2.5 hours. The mixture was filtered through Celite® and the solvent was evaporated under reduced pressure. The residue was triturated with dichloromethane to give 1-[((2R, 3R,4R)-2-{(1R)-1-[3,5-bis(trifluoromethyl)phenyl]ethoxy}tetrahydro-3-(4-fluorophenyl)-2H-pyran-4-yl)methyl]piperazinone (1.53 g, 94%).

A portion (107 mg) was dissolved in methanol (1.5 mL) and poured onto an SCX cartridge (Varian Bond Elut™; 10 mL/500 mg). The cartridge was washed with methanol (4×2 mL), then eluted with methanolic ammonia (2M, 2×2 mL). The solvent was evaporated under reduced pressure, the residue was dissolved in ethyl acetate and ethereal hydrogen chloride (1M, 200 μl, 0.200 mmol) was added. The solvent was evaporated under reduced pressure and the residue was dried in vacuo to give the title compound as a colorless solid (100 mg, 86%).

¹H NMR (360 MHz, CD₃OD) δ 1.33 (3H, d, J 6.6 Hz), 1.48 (1H, dq, J 12.1, 4.6 Hz), 1.70-1.74 (1H, m), 2.31-2.40 (1H, m), 2.44-2.49 (1H, m), 2.95 (1H, dd, J 13.8, 5.4 Hz), 3.12-3.20 (1H, m), 3.22-3.31 (2H, m), 3.35-3.43 (2H, m), 3.54-3.69 (3H, m), 4.10 (1H, dd, J 11.7, 3.2 Hz), 4.25 (1H, d, J 8.1 Hz), 5.01 (1H, q, J 6.4 Hz), 6.97 (2H, t, J 8.7 Hz), 7.15-7.19 (2H, m), 7.31 (2H, s), and 7.74 (1H, s). m/z (ES⁺) 291 (M+1-C₁₀H₈F₆O).

### EXAMPLE 2

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-methylpiperazinone Hydrochloride

Sodium triacetoxyborohydride (153 mg, 0.728 mmol) was added to a solution of 1-[((2R,3R,4R)-2-{(1R)-1-[3,5-bis(trifluoromethyl)phenyl]ethoxy}tetrahydro-3-(4-fluorophenyl)-2H-pyran-4-yl)methyl]piperazinone (Example 1; 100 mg, 0.182 mmol) and aqueous formaldehyde (38%, 52.5 μl, 0.728 mmol) in dichloroethane (10 mL) and the mixture was stirred at room temperature for 16 hours. Further aqueous formaldehyde (38%, 1.0 mL, 13.7 mmol) and sodium triacetoxyborohydride (200 mg, 0.943 mmol) were added and the mixture was stirred at room temperature for 24 hours. The solvent was evaporated under reduced pressure and dichloromethane (5 mL) and saturated aqueous sodium hydrogen carbonate (5 mL) were added. The layers were separated and the organic layer was poured onto an SCX cartridge (Varian Bond Elut™; 10 mL/500 mg). The cartridge was washed with methanol (4×2 mL), then eluted with methanolic ammonia (2M, 2×2 mL). The solvent was evaporated under reduced pressure, the residue was dissolved in ethyl acetate and ethereal hydrogen chloride (1M, 146 μl, 0.146 mmol) was added. The solvent was evaporated under reduced pressure and the residue was dried in vacuo to give the title compound (79.4 mg, 74%). ¹H NMR (400 MHz, CD₃OD) δ 1.33 (3H, d, J 6.6 Hz), 1.46 (1H, dq, J 12.3, 4.7 Hz), 1.66-1.70 (1H, m), 2.32-2.39 (1H, m), 2.42-2.49 (4H, m), 2.72-2.81 (1H, br), 2.82-2.89 (3H, m), 3.11-3.17 (2H, m), 3.27-3.40 (2H, m), 3.46-3.50 (1H, m), 3.56-3.66 (1H, m), 4.07-4.11 (1H, m), 4.25 (1H, d, J 8.2 Hz), 5.00 (1H, q, J 6.6 Hz), 6.94-6.98 (2H, m), 7.14-7.17 (2H, m), 7.31 (2H, s), and 7.73 (1H, s). m/z (ES⁺) 563 (M+1), 305 (M+1-C₁₀H₈F₆O).

### EXAMPLE 3

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-ethylpiperazinone Hydrochloride

Prepared from 1-[((2R,3R,4R)-2-{(1R)-1-[3,5-bis(trifluoromethyl)phenyl]ethoxy}-tetrahydro-3-(4-fluorophenyl)-2H-pyran-4-yl)methyl]piperazinone (Example 1) and acetaldehyde according to the method of Example 2. ¹H NMR (400 MHz, CD₃OD) δ 1.14 (3H, t, J 7.2 Hz), 1.33 (3H, d, J 6.6 Hz), 1.46 (1H, dq, J 12.4, 4.7 Hz), 1.69 (1H, dd, J 4.0, 2 Hz), 2.32-2.38 (1H, m), 2.43-2.48 (1H, m), 2.62-2.74 (3H, br), 2.88 (2H, dd, J 13.7, 4.9 Hz), 3.12-3.15 (2H, m), 3.22-3.39 (3H, m), 3.59 (1H, dt, J 12.2, 2.1 Hz), 4.07-4.11 (1H, m), 4.25 (1H, d, J 8.2 Hz), 5.00 (1H, q, J 6.5 Hz), 6.93-6.98 (2H, m), 7.14-7.18 (2H, m), 7.31 (2H, s), and 7.73 (1H, s). m/z (ES⁺) 577 (M+1), 319 (M+1-C₁₀H₈F₆O).

### EXAMPLE 4

### 1-[((2R,3R,4R)-2-{(1R)-1-[3,5-Bis(trifluoromethyl)phenyl]ethoxy}-tetrahydro-3-(4-fluorophenyl)-2H-pyran-yl)methyl]-4(1-methylethyl)piperazinone

Prepared from 1-[((2R,3R,4R)-2-{(1R)-1-[3,5-bis(trifluoromethyl)phenyl]ethoxy}-tetrahydro-3-(4-fluorophenyl)-2H-pyran-4-yl)methyl]piperazinone (Example 1) and acetone according to the method of Example 2. ¹H NMR (360 MHz, CD₃OD) δ 0.99 (6H, d, J 6.5 Hz), 1.33 (3H, d, J 6.6 Hz), 1.45 (1H, dq, J 12.3, 4.5 Hz), 1.65-4.70 (1H, m), 2.32-2.55 (4H, m), 2.63 (1H, m), 2.88-3.03 (4H, m), 3.12-3.18 (1H, m), 3.27-3.23 (1H, m), 3.59 (1H, dt, J 12.2, 2.1 Hz), 4.06-4.13 (1H, m), 4.23 (1H, d, J 8.1 Hz), 5.01 (1H, q, J 6.5 Hz), 6.92-6.96 (2H, m), 7.12-7.17 (2H, m), 7.31 (2H, s), and 7.73 (1H, s). m/z (ES⁺) 591 (M+1), 333 (M+1-C₁₀H₈F₆O).

### EXAMPLE 5

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-cyclohexylpiperazinone

Prepared from 1-[((2R,3R,4R)-2-{(1R)-1-[3,5-bis(trifluoromethyl)phenyl]ethoxy}-tetrahydro-3-(4-fluorophenyl)-2H-pyran-4-yl)methyl]piperazinone (Example 1) and cyclohexanone according to the method of Example 2. ¹H NMR (400 MHz, CD₃OD) δ 1.18-1.29 (6H, m), 1.33 (3H, d, J 6.6 Hz), 1.45 (1H, dq, J 12.4, 4.7 Hz), 1.60-1.69 (2H, m), 1.78-1.80 (4H, m), 2.20-2.25 (1H, m), 2.32-2.38 (1H, m), 2.40-2.47 (1H, m), 2.54-2.59 (1H, m), 2.90-3.01 (2H, m), 3.01-3.17 (2H, m), 3.27-3.32 (2H, m), 3.59 (1H, dt, J 12.2, 2.1 Hz), 4.06-4.12 (1H, m), 4.23 (1H, d, J 8.2 Hz), 5.00 (1H, q, J 6.6 Hz), 6.94 (2H, t, J 8.8 Hz), 7.12-7.17 (2H, m), 7.30 (2H, s), and 7.73 (1H, s). m/z (ES⁺) 373 (M+1-C₁₀H₈F₆O).

### EXAMPLE 6

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-(tetrahydropyran-4-yl)piperazinone

Prepared from 1-[((2R,3R,4R)-2-{(1R)-1-[3,5-bis(trifluoromethyl)phenyl]ethoxy}-tetrahydro-3-(4-fluorophenyl)-2H-pyran-4-yl)methyl]piperazinone (Example 1) and tetrahydro-4H-pyran-4-one according to the method of Example 2. ¹H NMR (360 MHz, CD₃OD,) δ 1.35 (3H, d, J 11.8 Hz), 1.39-1.51 (3H, m), 1.64-1.75 (3H, m), 2.33-2.48 (4H, m), 2.55-2.61 (1H, m), 2.90-3.03 (3H, m), 3.11-3.18 (1H, m), 3.28-3.39 (4H, m), 3.59 (1H, dt, J 11.0, 1.8 Hz), 3.94 (2H, dd, J 10.0, 3.3 Hz), 4.06-4.11 (1H, m), 4.24 (1H, d, J 7.3 Hz), 5.00 (1H, q, J 5.9 Hz), 6.92-6.97 (2H, m), 7.12-7.16 (2H, m), 7.31 (2H, s), and 7.73 (1H, s). m/z (ES⁺) 375 (M+1-C₁₀H₈F₆O).

### EXAMPLE 7

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4(1-methylpiperidin-4-yl)piperazinone

Prepared from 1-[((2R,3R,4R)-2-((1R)-1-[3,5-bis(trifluoromethyl)phenyl]ethoxy}-tetrahydro-3-(4-fluorophenyl)-2H-pyran-4-yl)methyl]piperazinone (Example 1) and 1-methyl-4-piperidinone according to the method of Example 2. ¹H NMR (360 MHz, CD₃OD) δ 1.33 (3H, d, J 5.9 Hz), 1.43-1.55 (5H, m), 1.64-1.68 (2H, br), 1.80-1.83 (2H, br), 2.12-2.28 (3H, m), 2.33 (3H, s), 2.42-2.46 (2H, m), 2.55-2.63 (1H, m), 2.91-3.02 (4H, m), 3.10-3.15 (2H, m), 3.56-3.62 (1H, m), 4.07 (1H, dd, J 10.7, 4.0 Hz), 4.23 (1H, d, J 7.3 Hz), 5.00 (1H, q, J 6.2 Hz), 6.94 (2H, t, J 7.8 Hz), 7.13-7.16 (2H, m), 7.30 (2H, s), and 7.73 (1H, s). m/z (ES⁺) 646 (M+1), 388 (M+1-C₁₀H₈F₆O).

### EXAMPLE 8

### 1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-phenylpiperazinone

A degassed flask was charged with tris(dibenzylideneacetone)dipalladium(0) (0.88 mg, 0.96 μmol), 2-(dicyclohexylphosphino)biphenyl (1.34 mg, 3.8 μmol) and sodium tert-butoxide (51.5 mg, 0.536 mmol). Toluene (1.54 mL) then bromobenzene (80.7 μl, 0.77 mmol) were added, followed by 1-[((2R,3R,4R)-2-{(1R)-1-[3,5-bis(trifluoromethyl)phenyl]ethoxy}tetrahydro-3-(4-fluorophenyl)-2H-pyran-4-yl)methyl]piperazinone (Example 1; 250 mg, 0.456 mmol) in toluene (2 mL) and the mixture was stirred at 80° C. for 18 hours. Further bromobenzene (80.7 μl, 0.77 mmol), sodium tert-butoxide (51.5 mg, 0.54 mmol), tris(dibenzylideneacetone)dipalladium(0) (0.88 mg, 0.96 μmol) and 2-(dicyclohexylphosphino)biphenyl (1.34 mg, 3.8 μmol) were added and the mixture was stirred at 80° C. for 3 hours. The mixture was cooled, diluted with ether (100 mL) and fitered through Celite™. The solvent was evaporated under reduced pressure and the residue was purified by flash column chromatography on silica gel, eluting with CH₂Cl₂/EtOAc/NH₃(Aq.) (84:15:1) to give the title compound (165 mg, 58%). ¹H NMR (360 MHz, CD₃OD) δ 1.33 (3H, d, J 6.6 Hz), 1.48 (1H, dq, J 12.2, 4.7 Hz), 1.64-1.69 (1H, m), 2.35-2.43 (1H, m), 2.44-2.49 (1H, m), 2.96 (1H, dd, J 13.6, 5.2 Hz), 3.13-3.41 (5H, m), 3.54-3.70 (3H, m), 4.07 (1H, dd, J 11.7, 3.2 Hz), 4.26 (1H, d, J 8.1 Hz), 5.00 (1H, q, J 6.5 Hz), 6.81-6.86 (3H, m), 6.94 (2H, t, J 8.7 Hz), 7.15-7.25 (4H, m), 7.31 (2H, s), and 7.73 (1H, s). m/z (ES⁺) 625 (M+1), 367 (M+1-C₁₀H₈F₆O).

### EXAMPLE 9

### 1-[((2R,3R,4R)-2-{(1R)-1-[3,5-Bis(trifluoromethyl)phenyl]ethoxy}-tetrahydro-3-(4-fluorophenyl)-2H-pyran-4yl)methyl]-4-(pyrid-3-yl)piperazinone

A degassed flask was charged with tris(dibenzylideneacetone)dipalladium(0) (3.2 mg, 3.5 μmol), (R)-(+)-2,2′-bis(diphenylphosphino)-1,1′-binapthyl (8.3 mg, 13.3 μmol) and sodium tert-butoxide (103 mg, 1.07 mmol). Toluene (2 mL), then 1-[((2R,3R,4R)-2-{(1R)-1-[3,5-bis(trifluoromethyl)phenyl]ethoxy}tetrahydro-3-(4-fluorophenyl)-2H-pyran-4-yl)methyl]piperazinone (Example 1; 250 mg, 0.456 mmol) in toluene (2 mL) were added, followed by 3-bromopyridine (103 μl, 1.06 mmol) and the mixture was stirred at 80° C. for 16 hours. The mixture was cooled, diluted with ether (100 mL) and filtered through Celite™. The solvent, was evaporated under reduced pressure and the residue was purified by flash column chromatography on silica gel, eluting with CH₂Cl₂/EtOAc/NH₃(Aq.) (792:8:1). The residue was dissolved in methanol (1.5 mL) and poured onto an SCX cartridge (Varian Bond Elut™; 10 mL/500 mg). The cartridge was washed with methanol (4×2 mL), then eluted with methanolic ammonia (2M, 2×2 mL). The solvent was evaporated under reduced pressure to give the title compound (131 mg, 46%). ¹H NMR (400 MHz, CDCl₃) δ 1.37 (3H, d, J 5.9 Hz), 1.53-1.63 (1H, m), 1.68-1.72 (1H, m), 2.21-2.29 (1H, m), 2.48-2.53 (1H, m), 2.97 (1H, dd, J 12.3, 4.4 Hz), 3.16-3.30 (3H, m), 3.37-3.42 (1H, m), 3.47-3.55 (2H, m), 3.69-3.80 (2H, m), 4.09-4.16 (2H, m), 4.92-4.97 (1H, m), 6.93-6.99 (2H, m), 7.04-7.10 (3H, m), 7.16-7.27 (3H, m), 7.68 (1H, s), 8.12 (1H, s), and 8.21 (1H, s). m/z (ES⁺) 368 (M+1-C₁₀H₈F₆O).

### EXAMPLE 10

### 4-[((2R,3R,4R)-2-{(1R)-1-[3,5-Bis(trifluoromethyl)phenyl]ethoxy}-tetrahydro-3-phenyl-2H-pyran-4-yl)methyl]piperazinone

Prepared from (2R,3R,4R)-2-{(1R)-1-[3,5-bis(trifluoromethyl)phenyl]ethoxy}-tetrahydro-3-phenyl-2H-pyran-4-carboxaldehyde (WO 00/56727-A1) and piperazinone according to the method of Example 2. ¹H NMR (360 MHz, CDCl₃) δ 7.66 (1H, s), 7.26-7.16 (5H, m), 7.01 (2H, m), 6.00 (1H, br s), 4.92 (1H, q, J 6.5 Hz), 4.19 (1H, d, J 8.3 Hz), 4.12 (1H, br d, J 12 Hz), 3.52 (1H, br t, J 12 Hz), 3.28-3.18 (2H, m), 3.07 (1H, d, J 16.5 Hz), 2.77 (1H, d, J 16.5 Hz), 2.52-2.34 (3H, m), 2.17-1.91 (4H, m), 1.45 (1H, m), and 1.36 (3H, d, J 6.5 Hz). m/z (ES⁺) 531 (M+1), 273 (M+1-C₁₀H₈F₆O).

### EXAMPLE 11

### 4[((2R,3R,4R)-2-{(1R)-1-[3,5-Bis(trifluoromethyl)phenyl]ethoxy}-tetrahydro-3-(4-fluorophenyl)-2H-pyran-4-yl)methyl]piperazinone

Prepared from (2R,3R,4R)-2-{(1R)-1-[3,5-bis(trifluoromethyl)phenyl]ethoxy}-tetrahydro-3-(4-fluorophenyl)-2H-pyran-4-carboxaldehyde (WO 03/022839-A1) and piperazinone according to the method of Example 2. ¹H NMR (400 MHz, CDCl₃) δ 7.68 (1H, s), 7.17 (2H, s), 7.01-6.92 (4H, m), 5.70 (1H, br s), 4.95 (1H, q, J 6.6 Hz), 4.14 (2H, m), 3.51 (1H, br t, J 12 Hz), 3.30-3.20 (2H, m), 3.08 (1H, d, J 16.5 Hz), 2.78 (1H, d, J 16.5 Hz), 2.48 (1H, m), 2.40 (2H, m), 2.15-1.85 (4H, m), 1.44 (1H, m), and 1.37 (3H, d, J 6.5 Hz). m/z (ES⁺) 549 (M+1), 291 (M+1-C₁₀H₈F₆O).

### EXAMPLE 12

### 4-[((2R,3R,4R)-2-{(1R)-1-[3,5-Bis(trifluoromethyl)phenyl]ethoxyl}-tetrahydro-3-(4-fluorophenyl)-2H-pyran-4-yl)methyl]-1-methylpiperazinone

Prepared from (2R,3R,4R)-2-{(1R)-1-[3,5-bis(trifluoromethyl)phenyl]ethoxy}-tetrahydro-3-(4-fluorophenyl)-2H-pyran-4-carboxaldehyde (WO 03/022839-A1) and 1-methylpiperazinone according to the method of Example 2. ¹H NMR (360 MHz, CDCl₃) δ 7.68 (1H, s), 7.17 (2H, s), 7.00-6.92 (4H, m), 4.95 (1H, q, J 6.6 Hz), 4.14 (2H, m), 3.50 (1H, br t, J 12 Hz), 3.24 (1H, m), 3.14 (1H, m), 3.08 (1H, d, J 16.2 Hz), 2.89 (3H, s), 2.74 (1H, d, J 16.2 Hz), 2.50-2.37 (3H, m), 2.11 (1H, m), 1.99-1.85 (3H, m), 1.42 (1H, m), and 1.36 (3H, d, J 6.6 Hz).

### EXAMPLE 13

### 4-[((2R,3R,4R)-2-{(1R)-1-[3,5-Bis(trifluoromethyl)phenyl]ethoxyl}-tetrahydro-3-(4-fluorophenyl)-2H-pyran-4-yl)methyl]-1-ethylpiperazinone

Prepared from (2R,3R,4R)-2-{(1R)-1-[3,5-bis(trifluoromethyl)phenyl]ethoxy}-tetrahydro-3-(4-fluorophenyl)-2H-pyran-4-carboxaldehyde (WO03/022839) and 1-ethylpiperazinone according to the method of Example 2. ¹H NMR (360 MHz, CDCl₃) δ 7.68 (1H, s), 7.17 (2H, s), 7.00-6.92 (4H, m), 4.94 (1H, q, J 6.6 Hz), 4.14 (2H, m), 3.50 (1H, br t, J 12 Hz), 3.36 (2H, q, J 7.2 Hz), 3.22 (1H, m), 3.13 (1H, m), 3.08 (1H, d, J 16.0 Hz), 2.74 (1H, d, J 16.0 Hz), 2.50-2.37 (3H, m), 2.12 (1H, m), 2.00-1.86 (3H, m), 1.44 (1H, m), 1.36 (3H, d, J 6.6 Hz), and 1.08 (3H, t, J 7.2 Hz). m/z (ES⁺) 577 (M+1), 319 (M+1-C₁₀H₈F₆O).

### EXAMPLE 14

### 4-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-1-phenylpiperazinone

Prepared from (2R,3R,4R)-2-{(1R)-1-[3,5-bis(trifluoromethyl)phenyl]ethoxy}-tetrahydro-3-(4-fluorophenyl)-2H-pyran-4-carboxaldehyde (WO 03/022839-A1) and 1-phenylpiperazinone (Tet. Lett. 1998, 39, 7459-7462) according to the method of Example 2. ¹H NMR (400 MHz, CDCl₃) δ 1.36 (3H, d, J 6.6 Hz), 1.44-1.52 (1H, m), 1.58-1.62 (1H, m), 1.59-2.10 (2H, m), 2.19 (1H, dd, J 12.2, 10.0 Hz), 2.44 (1H, dd, 11.1, 8.4 Hz), 2.53-2.58 (1H, m), 2.61-2.67 (1H, m), 3.26 (1H, d, J 16.4 Hz), 2.93 (1H, d, J 16.4 Hz), 3.45-3.65 (3H, m), 4.15 (1H, d, J 8.4 Hz), 4.11-4.17 (1H, m), 4.96 (1H, q, J 6.6 Hz), 6.93-7.03 (4H, m), 7.18 (2H, s), 7.19-7.27 (3H, m), 7.35-7.40 (2H, m), and 7.68 (1H, s). m/z (ES⁺) 625 (M+1), 367 (M+1-C₁₀H₈F₆O).

### EXAMPLE 15

### 4-((2R,3R,4R)-2-{1(1R)-1-[3,5-Bis(trifluoromethyl)phenyl]ethoxy}tetrahydro-3-(4-fluorophenyl)-2H-pyran-4-yl)methyl]-1-(pyrid-3-yl)piperazinone

Prepared from (2R,3R,4R)-2-{(1R)-1-[3,5-bis(trifluoromethyl)phenyl]ethoxy}-tetrahydro-3-(4-fluorophenyl)-2H-pyran-4-carboxaldehyde (WO 03/022839-A1) and 1-(3-pyridinyl)piperazinone (WO 01/44250-A1) according to the method of Example 2. ¹H NMR (360 MHz, CDCl₃) δ 1.36 (3H, d, J 6.6 Hz), 1.42-1.58 (1H, m), 1.58-1.62 (1H, m), 1.94-2.10 (2H, m), 2.20 (1H, dd, J 12.2, 10.0 Hz), 2.44 (1H, dd, 11.1, 8.4 Hz), 2.56-2.61 (1H, m), 2.64-2.70 (1H, m), 2.97 (1H, d, J 16.6 Hz), 3.27 (1H, d, J 16.6 Hz), 3.49-3.56 (2H, m), 3.63-3.68 (1H, m), 4.15 (1H, d, J 8.4 Hz), 4.14-4.18 (1H, m), 4.96 (1H, q, J 6.6 Hz), 6.93-7.03 (4H, m), 7.18 (2H, s), 7.31 (1H, dd, J 5.4, 4.8 Hz), 7.61-7.65 (1H, m), 7.68 (1H, s), 8.48 (1H, dd, J 4.8, 1.4 Hz), and 8.53 (1H, d, J 2.3 Hz). m/z (ES⁺) 626 (M+1), 368 (M+1-C₁₀H₈F₆O).

### EXAMPLE 16

### 4-[((2R,3S,4S)-2-{1(1R)-1-[3,5-Bis(trifluoromethyl)phenyl]ethoxy}-tetrahydro-3-(4-fluorophenyl)-2H-pyran-4-yl)methyl]piperazinone

Prepared from (2R,3S,4S)-2-{(1R)-1-[3,5-bis(trifluoromethyl)phenyl]ethoxy}-tetrahydro-3-(4-fluorophenyl)-2H-pyran-4-carboxaldehyde (Description 2) and piperazinone according to the method of Example 2. ¹H NMR (400 MHz, CDCl₈) δ 7.62 (1H, s), 7.18 (4H, m), 7.00 (2H, m), 5.80 (1H, br s), 4.89 (1H, q, J 6.6 Hz), 4.38 (1H, d, J 2.2 Hz), 3.99 (1H, br t, J 12 Hz), 3.76 (1H, br d, J 12 Hz), 3.37 (1H, m), 3.28 (1H, m), 3.25 (1H, d, J 16.6 Hz), 2.88 (1H, d, J 16.6 Hz), 2.65-2.40 (4H, m), 2.15-2.00 (3H, m), 1.47 (1H, m), and 1.46 (3H, d, J 6.6 Hz). m/z (ES+) 549 (M+1).

### EXAMPLE 17

### 4-[((2R,3S,4S)-2-{(1R)-1-[3,5-Bis(trifluoromethyl)]phenyl]ethoxy}-tetrahydro-3-(4-fluorophenyl)-2H-pyran-4-yl)methyl]-1-methylpiperazinone

Prepared from (2R,3S,4S)-2-{(R)-1-[3,5-bis(trifluoromethyl)phenyl]ethoxy}-tetrahydro-3-(4-fluorophenyl)-2H-pyran-4-carboxaldehyde Description 2) and 1-methylpiperazinone according to the method of Example 2. ¹H NMR (400 MHz, CDCl₈) δ 7.62 (1H, s), 7.18 (4H, m), 7.00 (2H, m), 4.89 (1H, q, J 6.6 Hz), 4.38 (1H, d, J 2.4 Hz), 3.98 (1H, br t, J 12 Hz), 3.75 (1H, br d, J 12 Hz), 3.34 (1H, m), 3.28 (1H, d, J 16.1 Hz), 3.20 (1H, m), 2.94 (3H, s), 2.83 (1H, d, J 16.1 Hz), 2.65-2.45 (4H, m), 2.15-1.95 (3H, m), 1.46 (3H, d, J 6.6 Hz), and 1.45 (1H, m). m/z (ES⁺) 563 (M+1).

### EXAMPLE 18

### 4-[((2R,3S,4S)-2-{(1R)-1-[3,5-Bis(trifluoromethyl)phenyl]ethoxy}-tetrahydro-3-(4-fluorophenyl)-2H-pyran-4-yl)methyl]-1-ethylpiperazinone

Prepared from (2R,3S,4S)-2-{(1R)-1-[3,5-bis(trifluoromethyl)phenyl]ethoxy}-tetrahydro-3-(4-fluorophenyl)-2H-pyran-4-carboxaldehyde (Description 2) and 1-ethylpiperazinone according to the method of Example 2. ¹H NMR (400 MHz, CDCl₃) δ 7.62 (1H, s), 7.17 (4H, m), 7.00 (2H, m), 4.89 (1H, q, J 6.6 Hz), 4.38 (1H, d, J 2.4 Hz), 3.98 (1H, br t, J 12 Hz), 3.75 (1H, br d, J 12 Hz), 3.41 (2H, q, J 7.2 Hz), 3.30 (1H, m), 3.27 (1H, d, J 16.2 Hz), 3.18 (1H, m), 2.83 (1H, d, J 16.2 Hz), 2.65-2.45 (4H, m), 2.15-1.95 (3H, m), 1.46 (3H, d, J 6.6 Hz), 1.45 (1H, m), and 1.12 (3H, t, J 7.2 Hz). m/z (ES⁺) 577 (M+1).

### EXAMPLE 19

### 4-[((2R,3R,4R)-2-{(1R)-1-[3,5-Bis(trifluoromethyl)phenyl]ethoxy}-tetrahydro-3-(3,4-difluorophenyl)-2H-pyran-4-yl)methyl]thiomorpholine 1,1-dioxide

Thiomorpholine 1,1-dioxide (15 mg, 0.11 mmol) was added to a solution of (2R,3R,4R)-2-{(1R)-1-[3,5-bis(trifluoromethyl)phenyl]ethoxy}tetrahydro-3-(3,4-difluorophenyl)-2H-pyran-4-carboxaldehyde (WO 02/16344-A1, 25 mg, 0.052 mmol) in dichloromethane (5 mL) and the mixture was stirred at room temperature for 1 hour. Sodium triacetoxyborohydride (16 mg, 0.075 mmol) was added and the mixture was stirred at room temperature for 24 hours. Saturated aqueous sodium hydrogen carbonate (5 mL) was added and the layers were separated. The aqueous layer was extracted with dichloromethane (5 mL), the organic phases were combined and the solvent was evaporated under reduced pressure. The residue dissolved in DMSO (1 mL) and purified by mass directed HPLC (Waters X-Terra™ MS C-8 19×100 mm; water/acetonitrile/0.1% TFA gradient; collected fractions were evaporated to dryness in Genevac™ HT-8 evaporator) to give the title compound as a colorless solid (19 mg, 61%). ¹H NMR (400 MHz, DMSO-d₆) δ 1.29 (4H, d, J 6.5 Hz), 1.86-1.75 (1H, m), 2.14-2.05 (2H, m), 2.42-2.26 (2H, m), 2.75-2.64 (1H, m), 2.96-2.86 (1H, m), 3.03 (2H, d, J 14.7 Hz), 4.04-3.97 (1H, m), 4.32 (1H, d, J 8.4 Hz), 5.06-4.99 (1H, m), 6.98 (1H, s), 7.26-7.17 (3H, m), 7.39 (2H, s), and 7.89 (1H, s). m/z (APci⁺) 602 (M+1).

### EXAMPLE 20

### 4-[((2R,3R,4R)-2-{(1R)-1-[3,5-Bis(trifluoromethyl)phenyl]ethoxy}-tetrahydro-3-phenyl-2H-pyran-4-yl)methyl]thiomorpholine 1.1-dioxide

Prepared from (2R,3R,4R)-2-{(1R)-1-[3,5-bis(trifluoromethyl)phenyl]ethoxy}-tetrahydro-3-phenyl-2H-pyran-4-carboxaldehyde (WO 00/56727-A1) and thiomorpholine 1,1-dioxide according to the method of Example 19. ¹H NMR (400 MHz, DMSO-d₆) δ 1.27 (4H, t, J 6.9 Hz), 1.84 (1H, d, J 13.2 Hz), 2.16-1.97 (3H, m), 2.34 (1H, t, J 9.5 Hz), 2.70 (2H, t, J 6.6 Hz), 2.87 (3H, s), 3.57 (1H, t, J 11.3 Hz) 4.01 (1H, dd, J 3.5, 11.3 Hz), 4.34 (1H, d, J 8.4 Hz), 5.04 (1H, d, J 6.5 Hz), 7.21-7.11 (5H, m), 7.37 (2H, s), and 7.85 (1H, s). m/z (APci⁺) 566 (M+1).

### EXAMPLE 21

### 1-[((2R,3R,4R)-2-{(1R)-1-[3,5-Bis(trifluoromethyl)phenyl]ethoxy}-tetrahydro-3-phenyl-2H-pyran-4-yl)methyl]-2-pyrrolidinone

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 2-pyrrolidinone according to the method of Description 1. ¹H NMR (400 MHz, CDCl₃) δ 1.35 (3H, d, J 6.7 Hz), 1.49-1.59 (1H, m), 1.63 (1H, m), 1.70-1.82 (2H, m), 2.07-2.27 (3H, m), 2.48 (1H, dd, J 8.2, 11.3 Hz), 2.83 (1H, dd, J 5.1, 14.1 Hz), 3.04 (1H, ddd, J 6.3, 8.2, 9.4 Hz), 3.12 (1H, ddd, J 6.3, 8.2, 9.4 Hz), 3.19 (1H, dd, J 8.2, 13.7 Hz), 3.50 (1H, dt, J 2.7, 13.1 Hz), 4.12 (1H, ddd, J 2.0, 4.2, 11.7 Hz), 4.15 (1H, d, J 8.2 Hz), 4.93 (1H, q, J 6.3 Hz), 7.05 (2H, m), 7.16 (2H, s), 7.20-7.27 (3H, m), and 7.66 (1H, s).

### EXAMPLE 22

### 1-[((2R,3R,4R)-2-{(1R)-1-[3,5-Bis(trifluoromethyl)phenyl]ethoxy}-tetrahydro-3-phenyl-2H-pyran-4-yl)methyl]-2,5-pyrrolidinedione

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 2,5-pyrrolidinedione according to the method of Description 1. ¹H NMR (400 MHz, CDCl₃) δ 1.33 (3H, d, J 6.7 Hz), 1.50 (1H, m), 1.62 (1H, m), 2.08-2.20 (4H, m), 2.46-2.59 (2H, m), 3.23 (1H, dd, J 5.9, 13.3 Hz), 3.44 (1H, dd, J 8.2, 13.7 Hz), 3.24 (1H, dd, J 5.9, 14.1 Hz), 3.27 (1H, d, J 6.3 Hz), 3.52 (1H, dt, J 2.3, 12.1 Hz), 4.05 (1H, d, J 7.8 Hz), 4.11 (1H, m), 4.88 (1H, q, J 6.7 Hz), 7.04 (2H, m), 7.08 (2H, s), 7.16-7.25 (3H, m), and 7.63 (1H, s).

### EXAMPLE 23

### 1-[((2R,3R,4R)-2-{(1R)-1-[3,5-Bis(trifluoromethyl)phenyl]ethoxy}-tetrahydro-3-phenyl-2H-pyran-4-yl)methyl]-2-imidazolidinone

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 2-imidazolidinone according to the method of Description 1. ¹H NMR (400 MHz, CDCl₃) δ 1.36 (3H, d, J 6.7 Hz), 1.45-1.55 (1H, m), 1.74 (1H, m), 2.01-2.14 (2H, m), 2.48 (1H, dd, J 8.2, 11.3 Hz), 2.76 (1H, dd, J 4.7, 14.1 Hz), 3.04 (1H, dd, J 8.6, 14.5 Hz), 3.09-3.24 (4H, m), 3.52 (1H, dt, J 2.0, 11.7 Hz), 4.07 (1H, s), 4.14 (1H, m), 4.17 (1H, d, J 8.2 Hz), 4.94 (1H, q, J 6.7 Hz), 7.05 (2H, m), 7.16 (2H, s), 7.20-7.25 (3H, m), and 7.66 (1H, s).

### EXAMPLE 24

### 1-[((2R,3R,4R)-2-{(1R)-1-[3,5-Bis(trifluoromethyl)phenyl]ethoxy}-tetrahydro-3-phenyl-2H-pyran-4-yl)methyl]-3-methyl-2-imidazolidinone

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 1-methyl-2-imidazolidinone according to the method of Description 1. ¹H NMR (400 MHz, CDCl₂) δ 1.35 (3H, d, J 6.7 Hz), 1.71-1.76 (1H, m), 2.04-2.11 (1H, m), 2.49 (1H, dd, J 8.2, 11.7 Hz), 2.68 (3H, s), 2.78 (1H, d, J 14.1 Hz), 2.95-3.10 (5H, m), 3.48-3.54 (1H, m), 4.11-4.14 (1H, m), 4.15 (1H, s), 4.17 (1H, s), 4.95 (1H, q, J 6.7 Hz), 7.04-7.06 (2H, m), 7.16 (2H, s), 7.22 (3H, dd, J 3.1, 3.1 Hz), and 7.65 (1H, s).

### EXAMPLE 25

### 3-[((2R,3R,4R)-2-{(1R)-1-[3,5-Bis(trifluoromethyl)phenyl]ethoxy}-tetrahydro-3-phenyl-2H-pyran-4-yl)methyl]-1-methyl-2,4-imidazolidinedione

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 1-methyl-2,4-imidazolidinedione according to the method of Description 1.

¹H NMR (400 MHz, CDCl₃) δ 1.34 (3H, d, J 6.7 Hz), 1.50 (1H, m), 1.64 (1H, m), 2.46-2.62 (2H, m), 2.73 (3H, s), 2.48 (1H, dd, J 8.2, 11.3 Hz), 2.76 (1H, dd, J 4.7, 14.1 Hz), 3.24 (1H, dd, J 5.9, 14.1 Hz), 3.27 (1H, d, J 6.3 Hz), 3.39 (1H, dd, J 7.8, 13.7 Hz), 3.52 (1H, dt, J 2.4, 12.1 Hz), 4.07 (1H, d, J 7.8 Hz), 4.12 (1H, dd, J 1.6, 4.3, 11.7 Hz), 4.88 (1H, q, J 6.7 Hz), 7.07 (2H, m), 7.10 (2H, s), 7.17-7.23 (3H, m), and 7.63 (1H, s).

### EXAMPLE 26

### 2-[((2R,3R,4R)-2-{(1R)-1-[3,5-Bis(trifluoromethyl)phenyl]ethoxy}-tetrahydro-3-phenyl-2H-pyran-4-yl)methyl]-5-ethyl-1,2,5-thiadiazolidine 1,1-dioxide

Prepared from (2R,3R,4R)-2-[(1R)-1-[3,5-bis(trifluoromethyl)phenyl]ethoxy]-tetrahydro-3-phenyl-2H-pyran-4-methyl benzenesulfonate (Description 6) and 2-ethyl-1,2,5-thiadiazolidine 1,1-dioxide (J. Med. Chem. 1994, 37, 3023-3032) according to the method of Description 1. ¹H NMR (400 MHz, CDCl₃) δ 1.20 (3H, t, J 7.4 Hz), 1.36 (3H, d, J 6.7 Hz), 1.55 (1H, m), 1.62 (1H, m), 2.02 (1H, m), 2.04-2.15 (1H, m), 2.45 (1H, dd, J 8.2, 11.4 Hz), 2.71-2.84 (2H, m), 2.95-3.07 (3H, m), 3.09-3.20 (3H, m), 3.55 (1H, dt, J 2.3, 12.1 Hz), 4.15 (1H, ddd, J 1.6, 4.3, 11.7 Hz), 4.19 (1H, d, J 7.8 Hz), 4.94 (1H, q, J 6.7 Hz), 7.05 (2H, m), 7.16 (2H, s), 7.21-7.26 (3H, m), and 7.65 (1H, s).

### EXAMPLE 27

### (5R or S)-5-((2R,3R,4R)-2-{(1R)-1-[3,5-Bis(trifluoromethyl)phenyl]ethoxy}-tetrahydro-3-phenyl-2H-pyran-4-yl)-2,4-imidazolidinedione

A solution of (2R,3R,4R)-2-{(1R)-1-[3,5-bis(trifiluoromethyl)phenyl]ethoxy}-tetrahydro-3-phenyl-2H-pyran-4-carboxaldehyde (WO 00/56727-A1; 350 mg, 0.78 mmol) and potassium cyanide (100 mg, 1.51 mmol) in methanol (5 mL) was stirred at room temperature for 30 minutes. Ammonium carbonate (750 mg, 7.8 mmol) and water (5 mL) were added and the mixture was stirred at 70° C. for 7 hours. The mixture was cooled and the solvent was evaporated under reduced pressure. Water was added and the mixture was acidified with hydrochloric acid (6M) (CAUTION: HCN evolution). The mixture was extracted with ethyl acetate, the combined organic fractions were dried (MgSO₄) and the solvent was evaporated under reduced pressure. The residue was purified by preparative thin layer chromatography on silica gel, eluting with CH₂Cl₂/MeOH (95:5). The residue was recrystallised from ethyl acetate to give the title compound (single diastereoisomer). ¹H NMR (400 MHz, DMSO-d₆) □ 1.20 (1H, m), 1.28 (3H, d, J 6.7 Hz), 1.39-1.52 (1H, m), 2.30 (1H, m), 2.65 (1H, dd, J 8.6, 12.5 Hz), 3.65 (1H, m), 4.01 (1H, dd, J 3.9, 11.4 Hz), 4.56 (1H, d, J 8.6 Hz), 5.05 (1H, q, J 6.7 Hz), 7.23 (5H, m), 7.40 (2H, s), 7.86 (1H, m), and 8.13 (1H, s).

### EXAMPLE 28

### (3R or S)-3-((2R,3R,4R)-2-{(1R)-1-[3,5-Bis(trifluoromethyl)phenyl]ethoxy}-tetrahydro-3-phenyl-2H-pyran-4-yl)-4-methylthiomorpholine 1,1-dioxide

Triethylamine (0.072 mL, 0.52 mmol) was added to a stirred, cooled (−20° C.) solution of (2R,3R,4R,αR or S)-α-{[(2-hydroxyethyl)sulfonyl]methyl}-2-{(1R)-1-[3,5-bis(trifluoromethyl)phenyl]ethoxy}tetrahydro-3-phenyl-2H-pyran-4-methanol and (2R,3R,4R, αS or R)-α-{[(2-hydroxyethyl)sulfonyl]methyl}-2-{(1R)-1-[3,5-bis(trifluoromethyl)phenyl]ethoxy}tetrahydro-3-phenyl-2H-pyran-4-methanol (Description 5; 1:1 mixture of alcohol epimers, 75 mg, 0.13 mmol) in dichloromethane (2 mL) and the mixture was stirred at −20° C. for 5 minutes. Methanesulfonyl chloride (0.03 mL, 0.388 mmol) was added slowly and the mixture was stirred at −20° C. for 20 minutes. Water (5 mL) was added and the mixture was extracted with dichloromethane (2×5 mL). The combined organic fractions were washed with aqueous citric acid (10%, 10 mL) then saturated aqueous sodium bicarbonate (10 mL), dried (MgSO₄) and the solvent was evaporated under reduced pressure. The residue was dissolved in methylamine (2M solution in methanol, 2 mL, 4 mmol), placed in a sealed tube and heated in a microwave oven at 130° C. for 10 minutes. The mixture was cooled and the solvent was evaporated under reduced pressure. The residue was dissolved in methanol (1.5 mL) and poured onto an SCX cartridge (Varian Bond Elut™; 10 mL/500 mg). The cartridge was washed with methanol (4×2 mL), then eluted with methanolic ammonia (2M, 2×2 mL). The solvent was evaporated under reduced pressure. The residue was purified by column chromatography on silica gel, eluting with CH₂Cl₂/MeOH (100:0 increasing to 98:2), to give the title compound (single diastereoisomer) as a pale cream-coloured solid (7.3 mg, 10%).

¹H NMR (500 MHz, CD₃OD) δ 1.32 (3H, d, J 6.6 Hz), 1.50 (1H, m), 1.72 (1H, dd, J 14.0, 2.4 Hz), 2.16 (1H, m), 2.21 (3H, s), 2.45-2.55 (3H, m), 2.87 (1H, d, J 12.0 Hz), 2.99 (2H, dd, J 7.4, 2.2 Hz), 3.05-3.12 (2H, m), 3.65 (1H, dt, J_{d} 11.4, Jₜ 2.2 Hz), 4.10-4.13 (1H, m), 4.41 (1H, d, J 7.5 Hz), 5.00 (1H, q, J 6.6 Hz), 7.13 (2H, dd, J 8.2, 1.6 Hz), 7.20-7.26 (3H, m), 7.31 (2H, s), and 7.71 (1H, s). m/z (ES⁺) 566 (M+1), 308 (M+1-C₁₀H₈F₆O).

### EXAMPLE 29

### 2-[((2R,3R,4R)-2-{(1R)-1-[3,5-Bis(trifluoromethyl)phenyl]ethoxy}-tetrahydro-3-phenyl-2H-pyran-4-yl)methyl]isothiazolidine 1,1-dioxide

3-Chloro-1-propanesulfonyl chloride (0.027 mL, 0.224 mmol) was added to a solution of (2R,3R,4R)-2-[(1R)-1-[3,5-bis(trifluoromethyl)phenyl]ethoxy]-tetrahydro-3-phenyl-2H-pyran-4-methylamine (Description 8; 100 mg, 0.224 mmol) and N-ethyldiisopropylamine (0.078 mL, 0.448 mmol) in 1,2-dichloroethane (5 mL) and the mixture was stirred at room temperature for 3 days. Water was added and the layers were separated. The organic fraction was dried (MgSO₄) and the solvent was evaporated under reduced pressure. The residue was purified by preparative thin layer chromatography on silica gel, eluting with EtOAc/hexane (50:50) and the residue was recrystallised from hexane. The solid was collected, dissolved in 1,2-dichloroethane and aqueous sodium hydroxide solution (50%) and tetra-n-butylammonium bromide (3 mg) were added. The mixture was stirred at room temperature for 2 hours. The layers were separated, the organic fraction was dried (MgSO₄) and the solvent was evaporated under reduced pressure. The residue was purified by preparative thin layer chromatography on silica gel, eluting with EtOAc/hexane (50:50) to give the title compound. ¹H NMR (400 MHz, CDCl₃) δ 1.36 (3H, d, J 6.7 Hz), 1.52 (1H, m), 1.95 (1H, m), 2.00-2.12 (1H, m), 2.19 (2H, m), 2.44 (1H, dd, J 8.2, 11.3 Hz), 2.68 (1H, dd, J 3.9, 14.1 Hz), 2.82 (1H, dd, J 9.0, 13.7 Hz), 2,87-3.13 (4H, m), 2.95-3.07 (3H, m), 3.09-3.20 (3H, m), 3.54 (1H, dt, J 2.4, 12.1 Hz), 4.15 (1H, ddd, J 1.6, 4.7, 12.1 Hz), 4.19 (1H, d, J 7.8 Hz), 4.94 (1H, q, J 6.7 Hz), 7.06 (2H, m), 7.16 (2H, s), 7.21-7.28 (3H, m), and 7.65 (1H, s).

1-21. (canceled)

22. A compound of the formula (I): 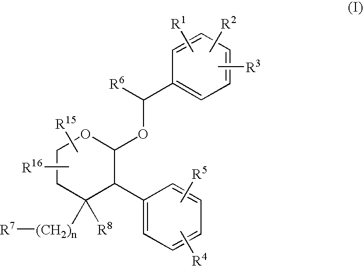
wherein:
R¹ is hydrogen, halogen, C₁₋₆alkyl, C₁₋₆alkoxy, fluoroC₁₋₆alkyl, fluoroC₁₋₆alkoxy, C₃₋₇cycloalkyl, C₃₋₇cycloalkylC₁₋₄alkyl, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} each independently represent hydrogen or C₁₋₄alkyl;
R² is hydrogen, halogen, C₁₋₆alkyl, fluoroC₁₋₆alkyl or C₁₋₆alkoxy substituted by C₁₋₄alkoxy;
R³ is hydrogen, halogen or fluoroC₁₋₆alkyl;
R⁴ is hydrogen, halogen, C₁₋₆alkyl, C₁₋₆alkoxy, fluoroC₁₋₆alkyl, fluoroC₁₋₆alkoxy, hydroxy, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy;
R⁵ is hydrogen, halogen, C₁₋₆alkyl, fluoroC₁₋₆alkyl or C₁₋₆alkoxy substituted by C₁₋₄alkoxy;
R⁶ represents hydrogen or a C₁₋₄alkyl group which is unsubstituted or substituted by a hydroxy group;
R⁷ represents a 5- or 6-membered carbonyl or sulfonyl containing cyclic group comprising from 0 to 3 nitrogen ring atoms, from 0 to 1 oxygen ring atom and from 0 to 1 sulfur ring, wherein said ring is unsubstituted or substituted at any substitutable position by one or more substituents selected from ═O, halogen, hydroxy, R¹¹, R¹², SR^{f}, SO₂R^{g}, COR^{a}, CO₂R^{a}, CONR⁹R¹⁰, -ZNR⁹R¹⁰, benzyl, C₁₋₄alkyl, hydroxyC₁₋₄alkyl, fluoroC₁₋₄alkyl, chloroC₁₋₄alkyl, C₁₋₄alkoxyC₁₋₄alkyl, C₃₋₇cycloalkyl, C₃₋₇cycloalkylC₁₋₄alkyl, C₃₋₇cycloalkoxy, C₃₋₇cycloalkoxyC₁₋₄alkyl, C₁₋₄alkoxy, fluoroC₁₋₄alkoxy, hydroxyC₁₋₄alkoxy, C₁₋₄alkoxyC₁₋₄alkoxy, aryl, arylC₁₋₄alkyl, heteroaryl, heteroarylC₁₋₄alkyl or a 5- or 6-membered ring containing in the ring one oxygen atom or N(C₁₋₆alkyl), wherein R^{f} is C₁₋₄alkyl or aralkyl or aryl and R^{g} is C₁₋₄alkyl, aryl, arylC₁₋₄alkyl or NR⁹R¹⁰;
R⁸ represents hydrogen, C₁₋₆alkyl, fluoroC₁₋₆alkyl, hydroxy, C₁₋₆alkoxy, hydroxyC₁₋₆alkyl NR⁹R¹⁰, CONR⁹R¹⁰ or SO₂R^{g};
R⁹ is hydrogen, C₁₋₄alkyl, C₃₋₇cycloalkyl, C₃₋₇cycloalkylC₁₋₄alkyl, fluoroC₁₋₄alkyl, C₂₋₄alkyl substituted by a C₁₋₄alkoxy or hydroxyl group, or R⁹ is a five membered or six membered nitrogen-containing heteroaromatic ring as previously defined;
R¹⁰ is hydrogen or C₁₋₄alkyl, C₃₋₇cycloalkyl, C₃₋₇cycloalkylC₁₋₄alkyl, fluoroC₁₋₄alkyl or C₂₋₄alkyl substituted by a C₁₋₄alkoxy or hydroxyl group;
or R⁹, R¹⁰ and the nitrogen atom to which they are attached form a heteroaliphatic ring of 4 to 7 ring atoms, unsubstituted or substituted by one or two groups selected from hydroxy, COR^{e}, CO₂R^{e}, C₁₋₄alkyl unsubstituted or substituted by a C₁₋₄alkoxy or hydroxyl group, or C₁₋₄alkoxy unsubstituted or substituted by a C₁₋₄alkoxy or hydroxyl group, or a five membered or six membered nitrogen-containing heteroaromatic ring as previously defined, or said heteroaliphatic ring is substituted by a spiro-fused lactone ring, and said heteroaliphatic ring optionally containing a double bond, which heteroaliphatic ring may contain an oxygen or sulphur ring atom, a group S(O) or S(O)₂ or a second nitrogen atom which will be part of a NH or NR^{d} moiety, where R^{d} is C₁₋₄alkyl unsubstituted or substituted by hydroxy or C₁₋₄alkoxy;
or R⁹, R¹⁰ and the nitrogen atom to which they are attached form a non-aromatic azabicyclic ring system of 6 to 12 ring atoms;
or R⁹, R¹⁰ and the nitrogen atom to which they are attached form a heteroaliphatic ring of 4 to 7 ring atoms to which is fused a benzene ring or a five membered or six membered nitrogen-containing heteroaromatic ring optionally containing 1, 2 or 3 additional heteroatoms selected from N, O and S;
R¹¹ and R¹² each independently represent hydrogen, hydroxy, COR^{e}, CO₂R^{e}, C₁₋₄alkyl unsubstituted or substituted by a C₁₋₄alkoxy or hydroxyl group, or C₁₋₄alkoxy unsubstituted or substituted by a C₁₋₄alkoxy or hydroxyl group;
or, when they are attached to the same carbon atom, R¹¹ and R¹² may together represent ═O, ═CHCO₂R^{a}, —O(CH₂)ₘO—, —CH₂O(CH₂)ₖ—, —CH₂OCH₂C(O)—, —CH₂OCH₂CH(OH)—, —CH₂OCH₂C(CH₃)₂—, —CH₂OC(CH₃)₂CH₂—, —C(CH₃)₂OCH₂CH₂—, —CH₂C(O)OCH₂—, —OC(O)CH₂CH₂—, —C(O)OCH₂CH₂—, —C(O)OC(CH₃)₂CH₂—, —C(O)OCH₂C(CH₃)₂—, —OCH₂(CH₂)ₖ—, —OC(CH₃)₂CH₂CH₂—, —OCH₂C(CH₃)₂CH₂—, —OCH₂CH₂C(CH₃)₂—, —OCH₂CH═CHCH₂—, —OCH₂CH(OH)CH₂CH₂—, —OCH₂CH₂CH(OH)CH₂—, —OCH₂C(O)CH₂CH₂—, —OCH₂CH₂C(O)CH₂—, or a group of the formula: 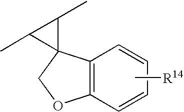
or, where they are attached to adjacent carbon atoms, R¹¹ and R¹² may together represent —OCH₂CH₂— or —OCH₂CH(OH)—, or R¹¹ and R¹² may together form a fused benzene ring;
or, R¹¹ and R¹² together form a C₁₋₂alkylene bridge across the pyrrolidine, piperidine, morpholine or piperazine ring to which they are attached;
R¹³ represents hydrogen, phenyl, benzyl, pyridyl, tetrahydropyranyl, piperidinyl, N-substituted piperidinyl (where the N-substituent is C₁₋₆alkyl), C₁₋₄alkyl, C₃₋₇cycloalkyl, C₃₋₇cycloalkylC₁₋₄alkyl, —SO₂C₁₋₄alkyl or C₂₋₄alkyl substituted by a C₁₋₄alkoxy or hydroxyl group;
R¹⁴ represents hydrogen, halogen, hydroxy, C₁₋₄alkyl, hydroxyC₁₋₄alkyl or fluoroC₁₋₄alkyl;
R¹⁵ and R¹⁶ each independently represent hydrogen, halogen, C₁₋₆alkyl, CH₂OR^{c}, oxo, CO₂R^{a} or CONR^{a}R^{b} where R^{a} and R^{b} are as previously defined and R^{c} represents hydrogen, C₁₋₆alkyl or phenyl;
Z represents a bond, C₁₋₆alkylene or C₃₋₆cycloalkylene;
k is 1, 2 or 3;
m is 1 or 2; and
n is zero, 1 or 2;
with the proviso that when n is zero and R⁸ is hydrogen, R⁷ does not represent a C-linked nitrogen-containing ring of the formula: 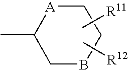
wherein:
A represents NR¹³, and B represents a bond, CH₂, NR¹³ or O, wherein one or both hydrogen atoms in said CH₂ moiety may be replaced with one or both of R¹¹ and R¹², or alternatively, one of the hydrogen atoms in said CH₂ moiety together with a hydrogen atom from an adjacent carbon are replaced by a double bond; or A is O, and B is NR¹³; and R¹¹ and R¹² together represent ═O;
and pharmaceutically acceptable salts thereof.

23. The compound of claim 22 wherein R¹ is hydrogen, C₁₋₄alkyl, C₁₋₄alkoxy, halogen or CF₃.

24. The compound of claim 22 wherein R² is hydrogen, C₁₋₄alkyl, C₁₋₄alkoxy, halogen or CF₃.

25. The compound of claim 22 wherein R³ is hydrogen, fluorine, chlorine or CF₃.

26. The compound of claim 22 wherein R⁴ is hydrogen or fluorine.

27. The compound of claim 22 wherein R⁵ is hydrogen, fluorine, chlorine or CF₃.

28. The compound of claim 22 wherein R is C₁₋₄alkyl optionally substituted by hydroxy.

29. The compound of claim 22 wherein R⁷ is a cyclic group selected from the group consisting of: 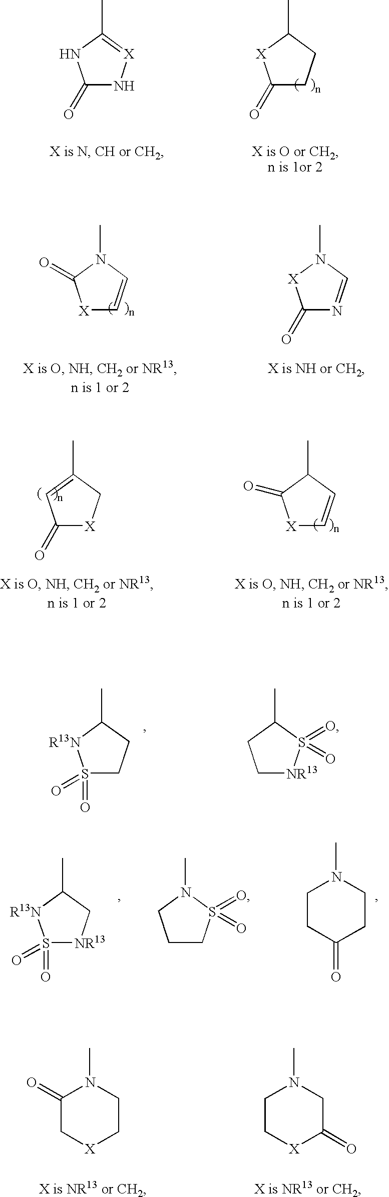 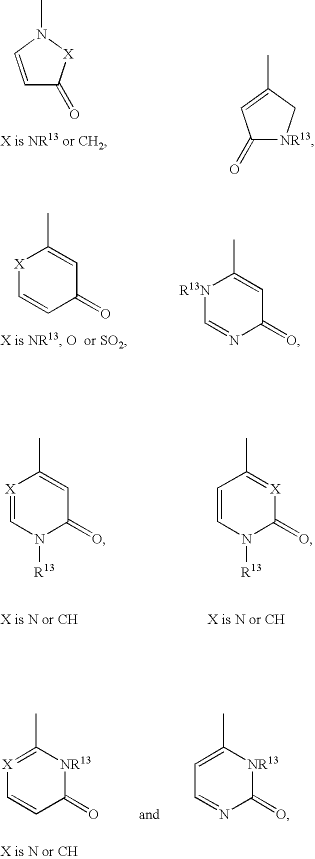
wherein any of said cyclic groups is unsubstituted or substituted by one or more groups as defined in claim 22.

30. The compound of claim 22 wherein R⁷ is a cyclic group selected from the group consisting of: 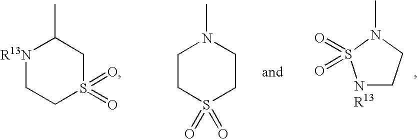
wherein any of said cyclic groups is unsubstituted or substituted by one or more groups as defined in claim 1.

31. The compound of claim 22 wherein R⁸ is hydrogen or methyl.

32. The compound of claim 22 wherein R¹² is hydrogen, hydroxy, C₁₋₂alkyl substituted by hydroxy, C₁₋₄alkoxy or CO₂R^{e}, where R^{e} is hydrogen, methyl ethyl or benzyl.

33. The compound of claim to 11 wherein R¹³ represents hydrogen, methyl or ethyl.

34. The compound of claim 22 wherein R¹⁵ is hydrogen and R¹⁶ is hydrogen.

35. The compound of claim 22 wherein n is zero or 1.

36. The compound of claim 22 of the formula (Ia): 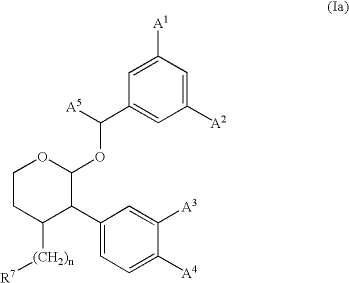
wherein:
A¹ is fluorine or CF₃;
A² is fluorine or CF₃;
A³ is fluorine or hydrogen;
A⁴ is fluorine or hydrogen;
A⁵ is methyl;
or a pharmaceutically acceptable salt thereof.

37. A compound which is selected from the group consisting of:
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]piperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-methylpiperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-ethylpiperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-(1-methylethyl)piperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-cyclohexylpiperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-(tetrahydropyran-4-yl)piperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-(1-methylpiperidin-4-yl)piperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-phenylpiperazinone;
1-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-4-(pyrid-3-yl)piperazinone;
4-[((2R,3R,4R)-2-{(1R)-1-[3,5-Bis(trifluoromethyl)phenyl]ethoxy}-tetrahydro-3-phenyl-2H-pyran-4-yl)methyl]piperazinone;
4-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-1-methylpiperazinone;
4-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-1-ethylpiperazinone;
4-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-1-phenylpiperazinone;
4-[((2R,3R,4R)-2-{(1R)-1-[3,5-Bis(trifluoromethyl)phenyl]ethoxy}-tetrahydro-3-(4-fluorophenyl)-2H-pyran-4-yl)methyl]-1-(pyrid-3-yl)piperazinone;
4-[((2R,3S,4S)-2-{(1R)-1-[3,5-Bis(trifluoromethyl)phenyl]ethoxy}-tetrahydro-3-(4-fluorophenyl)-2H-pyran-4-yl)methyl]piperazinone;
4-[((2R,3S,4S)-2-{(1R)-1-[3,5-Bis(trifluoromethyl)phenyl]ethoxy}-tetrahydro-3-(4-fluorophenyl)-2H-pyran-4-yl)methyl]-1-methylpiperazinone;
4-[((2R,3S,4S)-2-{(1R)-1-[3,5-Bis(trifluoromethyl)phenyl]ethoxy}-tetrahydro-3-(4-fluorophenyl)-2H-pyran-4-yl)methyl]-1-ethylpiperazinone;
4-[((2R,3R,4R)-2-{(1R)-1-[3,5-Bis(trifluoromethyl)phenyl]ethoxy}-tetrahydro-3-(3,4-difluorophenyl)-2H-pyran-4-yl)methyl]thiomorpholine 1,1-dioxide;
4-[((2R,3R,4R)-2-{(1R)-1-[3,5-Bis(trifluoromethyl)phenyl]ethoxy}-tetrahydro-3-phenyl-2H-pyran-4-yl)methyl]thiomorpholine 1,1-dioxide;
1-[((2R,3R,4R)-2-{(1R)-1-[3,5-Bis(trifluoromethyl)phenyl]ethoxy}-tetrahydro-3-phenyl-2H-pyran-4-yl)methyl]-2-pyrrolidinone;
1-[((2R,3R,4R)-2-{(1R)-1-[3,5-Bis(trifluoromethyl)phenyl]ethoxy}-tetrahydro-3-phenyl-2H-pyran-4-yl)methyl]-2,5-pyrrolidinedione;
1-[((2R,3R,4R)-2-{(1R)-1-[3,5-Bis(trifluoromethyl)phenyl]ethoxy}-tetrahydro-3-phenyl-2H-pyran-4-yl)methyl]-2-imidazolidinone;
1-[((2R,3R,4R)-2-{(1R)-1-[3,5-Bis(trifluoromethyl)phenyl]ethoxy}-tetrahydro-3-phenyl-2H-pyran-4-yl)methyl]-3-methyl-2-imidazolidinone;
3-[((2R,3R,4R)-2-{(1R)-1-[3,5-Bis(trifluoromethyl)phenyl]ethoxy}-tetrahydro-3-phenyl-2H-pyran-4-yl)methyl]-1-methyl-2,4-imidazolidinedione;
2-[((2R,3R,4R)-2-{(1R)-1-[3,5-Bis(trifluoromethyl)phenyl]ethoxy}-tetrahydro-3-phenyl-2H-pyran-4-yl)methyl]-5-ethyl-1,2,5-thiadiazolidine 1,1-dioxide;
(5R or S)-5-((2R,3R,4R)-2-{(1R)-1-[3,5-Bis(trifluoromethyl)phenyl]ethoxy}-tetrahydro-3-phenyl-2H-pyran-4-yl)-2,4-imidazolidinedione;
(3R or S)-3-((2R,3R,4R)-2-{(1R)-1-[3,5-Bis(trifluoromethyl)phenyl]ethoxy}-tetrahydro-3-phenyl-2H-pyran-4-yl)-4-methylthiomorpholine 1,1-dioxide;
2-[((2R,3R,4R)-2-{(1R)-1-[3,5-Bis(trifluoromethyl)phenyl]ethoxy}-tetrahydro-3-phenyl-2H-pyran-4-yl)methyl]isothiazolidine 1,1-dioxide;
or a pharmaceutically acceptable salt thereof.

38. A pharmaceutical composition comprising the compound of claim 22 and at least one pharmaceutically acceptable carrier or excipient.

39. A method for the treatment of pain or inflammation, migraine, emesis, postherpetic neuralgia, depression or anxiety, which method comprises administration to a patient in need thereof of a therapeutically effective amount of the compound of claim 22.

40. A method for the prevention of pain or inflammation, migraine, emesis, postherpetic neuralgia, depression or anxiety, which method comprises administration to a patient in need thereof of a therapeutically effective amount of the compound of claim 22.
